(19) United States
(12) Patent Application Publication
(10) Pub. No.: US 2026/0041514 A1
(43) Pub. Date: Feb. 12, 2026

(54) INTRAVASCULAR ROBOT DEVICES, SYSTEMS, AND METHODS OF USE

(71) Applicant: Avni AGARWAL, Mountain House, CA (US)

(72) Inventor: Avni AGARWAL, Mountain House, CA (US)

(21) Appl. No.: 19/200,470

(22) Filed: May 6, 2025

Related U.S. Application Data
(60) Provisional application No. 63/679,990, filed on Aug. 6, 2024.

(51) Int. Cl. A61B 34/00; A61B 17/22; A61B 34/30; A61F 2/95

(52) U.S. Cl. CPC A61B 34/73; A61B 17/22012; A61F 2/95; A61B 2017/22001; A61B 2034/303

(57) ABSTRACT

Provided herein are intravascular robots and corresponding system that may navigate the robot through one or more blood vessels and treat intravascular disease of the one or more blood vessels.

### CROSS-REFERENCE

This application claims the benefit of U.S. Provisional Application No. 63/679,990, filed Aug. 6, 2024, which is incorporated herein by reference in its entirety.

### BACKGROUND OF THE INVENTION

Current angioplasty and atherectomy procedures to treat an intravascular plaque-based narrowing of the blood vessel involve costly and invasive procedures wherein a guidewire and catheter external to the subject are inserted to guide a device to the region of the intravascular plaque within the blood vessel. Such procedures are invasive and may cause complications e.g., damage to the blood vessel wall caused by the catheter or guide wire or the guide wire becoming stuck, breaking off, kinking, or looping intravascularly. Traditional angioplasty procedures require multiple insertions of a lengthy catheter for prolonged periods of time in a subject's blood vessel that increases the risk of infection for the subject from microbes that colonize the catheter. Moreover, complications with regards to atherectomy procedures arise when a fragment of the intravascular plaque breaks off during the procedure and blocks a downstream blood vessel leading to a stroke. Thus, there exists an unmet need to further reduce common complications associated with the lifesaving and prolonging procedures of angioplasty and atherectomy to restore a subject's occluded or restricted blood flow caused by intravascular plaque.

### SUMMARY OF THE INVENTION

Aspects of the invention described herein comprise a low-cost intravascular device that addresses the above-mentioned complications of angioplasty and/or atherectomy procedures. In some embodiments, the intravascular device does not couple to and/or utilize a guidewire to navigate through a blood vessel. By circumventing the use of a guidewire, the invention described herein avoids the aforementioned shortcomings and complications of puncturing, damaging, and/or perforating a wall of a subject's blood vessel or blocking a downstream blood vessel with a portion of the guidewire that has broken off. In some cases, the one or more magnetic field generators external to the blood vessel of a subject, and the one or more magnets coupled to and/or disposed in the intravascular device, described elsewhere herein, may guide and/or translate the intravascular device without the use of a guide wire. In some embodiments, the intravascular device described herein, may reduce and/or mitigate complications that arise from inserting, removing, and re-inserting one or more intravascular catheters into a subject's blood vessel by combining the functionality of the one or more intravascular catheters into a single intravascular device. In some embodiments, the angioplasty intravascular device described elsewhere herein may combine the function of two separate angioplasty catheters: a catheter used to expand the narrowed blood vessel, and a catheter to deploy a stent or other mesh structures to maintain the enlarged opening of the blood vessel. In some embodiments, the intravascular device described elsewhere herein, combines the two separate catheters functionality into a single device, thereby reducing risk of infection of the subject that may otherwise occur through the insertion of two separate catheters into the subject's blood vessel. In some embodiments, the intravascular device, described elsewhere herein, may minimize the likelihood of downstream blood vessels blockages by plaque fragments generated from traditional atherectomy procedures by reducing the surface area and/or volume of the cutting elements of the intravascular atherectomy device. The reduced surface area and/or volume of the cutting elements may prevent the likelihood of removing and/or breaking off a larger fragment of intravascular plaque that may cause a downstream blockage.

Aspects of the disclosure describe, an intravascular device, comprising: a first curved body coupled to a rod at a first end of the rod, wherein a magnet is disposed within the first curved body; a mesh structure concentric with the rod, wherein the mesh structure comprises an inner surface and an outer surface; a deformable body adjacent to the inner surface of the mesh structure; and a second curved body coupled to a second end of the rod, wherein the second curved body is concentric with the rod, and wherein the second curved body applies a force to the deformable body thereby expanding the mesh structure when the second curved body is translated toward the first curved body at the first end of the rod. In some embodiments, the first curved body, the second curved body, or a combination thereof, comprise a hemisphere curved body. In some embodiments, the deformable body encapsulates a fluid. In some embodiments, the fluid comprises an osmolarity or density of about an osmolarity of mammalian blood or about a density of mammalian blood. In some embodiments, the deformable body comprises a balloon. In some embodiments, the mesh structure comprises a stent. In some embodiments, the stent comprises a drug eluting stent. In some embodiments, the first curved body, the second curved body, or a combination thereof comprises a flat surface. In some embodiments, the flat surface contacts an external surface of the deformable body. In some embodiments, a third body is coupled to the first curved body, the second curved body, or a combination thereof, and wherein the third body extends orthogonally away from a surface of the first curved body, the second curved body, or a combination thereof. In some embodiments, the third body comprises a tail of the first curved body, the second curved body, or a combination thereof. In some embodiments, the third body is coupled to the first curved body, the second curved body, or a combination thereof, at an apex of a curved surface of the first curved body, the second curved body, or a combination thereof. In some embodiments, the third body comprises a length of about a diameter of the first curved body or a diameter of the second curved body. In some embodiments, the rod comprises a threaded rod. In some embodiments, the second curved body comprises an opening, wherein the opening of the second curved body is concentric with the rod. In some embodiments, the opening comprises a threaded surface reversibly mated to the threaded rod. In some embodiments, the second curved body comprises a threaded mounting interface disposed within the second curved body, wherein the threaded mounting interface comprises an opening concentric or cordial with the opening of the second curved body, the rod, or a combination thereof. In some embodiments, the opening of the threaded mounting interface comprises a threaded surface reversibly mated to the threaded rod. In some embodiments, the first curved body, the second curved body, or a combination thereof, is made of a biocompatible material. In some embodiments, the biocompatible material comprises a biocompatible plastic or silicon. In some embodiments, the deformable body comprises a material that changes from a first diameter to a second diameter when the second curved body applies a force to the deformable body, and wherein the first diameter is less than the second diameter. In some embodiments, the deformable body changes from the second diameter to the first diameter when the second curved body is translated away from the first curved body. In some embodiments, the material of the deformable body comprises a heat set material, plastic, thermoplastic polymer, or a combination thereof. In some embodiments, the first curved body, the second curved body, the rod, the mesh structure, the deformable body, or any combination thereof, are not coupled to a guide wire. In some embodiments, the first curved body is spaced a distance of up to about 15 millimeters from the second curved body. In some embodiments, a first pole and a second pole of the magnet are orthogonal to a central axis of the rod. In some embodiments, the magnet is coupled to the rod, and wherein the magnet, the rod, or a combination thereof, rotate within the first curved body. In some embodiments, the magnet coupled to the rod is concentric with a washer disposed within the first curved body.

Aspects of the disclosure describe an intravascular device, comprising: a first curved body coupled to a second curved body; a magnet disposed within the first curved body, the second curved body, or a combination thereof; and a third body coupled to the first curved body, the second curved body, or a combination thereof, wherein the third body extends orthogonally away from a surface of the first curved body, a surface of the second curved body, or a combination thereof, and wherein the third body comprises a length of about a diameter of the first curved body coupled to the second curved body. In some embodiments, the first curved body, the second curved body, or a combination thereof comprises a hemisphere curved body. In some embodiments, the first curved body, the second curved body, or a combination thereof, are hollow. In some embodiments, the third body comprises a tail of the first curved body, the second curved body, or a combination thereof. In some embodiments, the third body comprises a helical body. In some embodiments, the third body comprises a rigid body. In some embodiments, the first curved body, the second curved body, or a combination thereof, are made of a biocompatible material. In some embodiments, the third body comprises a coil. In some embodiments, the surface of the first curved body, the surface of the second curved body, or a combination thereof, comprises a surface orthogonal to the first curved body, the second curved body, or a combination thereof. In some embodiments, the first curved body, the second curved body, or a combination thereof, are made of metal. In some embodiments, the first curved body, the second curved body, the third body, or any combination thereof, are not coupled to a guide wire. In some embodiments, the magnet is fixed within the first curved body, the second curved body, or a combination thereof.

Aspects of the disclosure describe a system for vibrating an intravascular device, comprising: a curved body comprising: a first curved body coupled to a rod at a first end of the rod, wherein a magnet is disposed within the first curved body; a mesh structure concentric with the rod, wherein the mesh structure comprises an inner surface and an outer surface; a deformable body adjacent to the inner surface of the mesh structure; and a second curved body coupled to a second end of the rod, wherein the second curved body is concentric with the rod; and a magnetic field generator, wherein the magnetic field generator provides an alternating magnetic field to the magnet disposed within the first curved body, thereby vibrating the first curved body, the second curved body, or a combination thereof, wherein the curved body is provided to a blood vessel of a subject, and wherein the magnetic field generator is external to the blood vessel of the subject. In some embodiments, the rod comprises a threaded rod. In some embodiments, the second curved body comprises an opening, wherein the opening of the second curved body is concentric with the rod. In some embodiments, the opening of the second curved body comprises a threaded surface reversibly mated to the threaded rod. In some embodiments, the second curved body comprises a threaded mounting interface disposed within the second curved body, and wherein the threaded mounting interface comprises an opening concentric or cordial with the opening of the second curved body, the rod, or a combination thereof. In some embodiments, the first curved body, the second curved body, or a combination thereof, comprise a hemisphere curved body. In some embodiments, the deformable body encapsulates a fluid. In some embodiments, the fluid comprises an osmolarity or density of about an osmolarity of mammalian blood or about a density of mammalian blood. In some embodiments, the deformable body comprises a balloon. In some embodiments, the deformable body comprises a material that changes from a first diameter to a second diameter when the second curved body applies a force to the deformable body, and wherein the first diameter is less than the second diameter. In some embodiments, the deformable body changes from the second diameter to the first diameter when the second curved body is translated away from the first curved body. In some embodiments, the material of the deformable body comprises a heat set material, plastic, thermoplastic polymer, or a combination thereof. In some embodiments, the mesh structure comprises a stent. In some embodiments, the stent comprises a drug eluting stent. In some embodiments, the first curved body, the second curved body, or a combination thereof comprises a flat surface. In some embodiments, the flat surface of the first curved body, the flat surface of the second curved body, or a combination thereof, contacts an outer surface of the deformable body. In some embodiments, the first curved body, the second curved body, or a combination thereof, is made of a biocompatible material. In some embodiments, the biocompatible material comprises a biocompatible plastic or silicon material. In some embodiments, the first curved body, the second curved body, the third body, or a combination thereof, are not coupled to a guidewire. In some embodiments, the first curved body is spaced a distance of up to about 15 millimeters from the second curved body.

Aspects of the disclosure describe a system for vibrating an intravascular device, comprising: a curved body comprising: a first curved body coupled to a second curved body; a magnet disposed within the first curved body, the second curved body, or a combination thereof; and a third body coupled to the first curved body, the second curved body, or a combination thereof, wherein the third body extends orthogonally away from a surface of the first curved body, a surface of the second curved body, or a combination thereof, and wherein the third body comprises a length of up to about a diameter of the first curved body coupled to the second curved body; and a magnetic field generator, wherein the magnetic field generator provides an alternating magnetic field to the magnet disposed within the first curved body, the second curved body, or a combination thereof, thereby vibrating the first curved body, the second curved body, or a combination thereof, wherein the curved body is provided to a blood vessel of a subject, wherein the magnetic field generator is external to the blood vessel of the subject. In some embodiments, the first curved body, the second curved body, or a combination thereof, comprise a hemisphere curved body. In some embodiments, the third body comprises a tail of the first curved body, the second curved body, or a combination thereof. In some embodiments, the third body is coupled to the first curved body, the second curved body, or a combination thereof, at an apex of a curved surface of the first curved body, the second curved body, or a combination thereof. In some embodiments, the first curved body, the second curved body, or a combination thereof, is made of a biocompatible material. In some embodiments, the biocompatible material comprises a biocompatible plastic or silicon material. In some embodiments, the first curved body, the second curved body, the third body, or a combination thereof, are not coupled to a guidewire. In some embodiments, the alternating magnetic field comprises a rotating magnetic field, and wherein when the rotating magnetic field is applied to the magnet, the third body propels or translates the first curved body, the second curved body, and the magnet within the blood vessel of the subject. In some embodiments, the magnet is fixed within the first curved body, the second curved body, or a combination thereof.

Aspects of the disclosure describe a method of fracturing intravascular plaque, comprising: vibrating a curved body in an inner lumen of a blood vessel of a subject with an alternating magnetic field provided external to the blood vessel, wherein the curved body comprises a magnet disposed within the curved body, wherein the curved body is not coupled to a guidewire, wherein the curved body contacts a surface of the inner lumen of the blood vessel adjacent to a blood vessel plaque, and wherein the curved body is vibrated at one or more frequencies in the blood vessel; and fracturing the blood vessel plaque when the vibrating curved body contacts the surface of the inner lumen of the blood vessel adjacent to the blood vessel plaque. In some embodiments, the curved body comprises a first curved body coupled to a second curved body. In some embodiments, the first curved body and the second curved body are coupled to a rod. In some embodiments, the first curved body is disposed at a first end of the rod, and wherein the second curved body is disposed at a second end of the rod. In some embodiments, the rod comprises a threaded region on a surface of the rod. In some embodiments, the first curved body, the second curved body, or a combination thereof, comprise a curved opening concentric with the rod. In some embodiments, a surface of the curved opening of the second curved body comprises a threaded region reversibly mated to the threaded region of the surface of the rod. In some embodiments, the rod is concentric with the first curved body, the second curved body, or a combination thereof. In some embodiments, a surface of the rod is in contact with a surface of the deformable body. In some embodiments, the first curved body, the second curved body, or a combination thereof, comprises a flat surface. In some embodiments, the flat surface of the first curved body, the flat surface of the second curved body, or a combination thereof, is in contact with an outer surface of the deformable body. In some embodiments, an outer surface of the deformable body is in contact with a mesh structure, and wherein the mesh structure is concentric with the rod. In some embodiments, the mesh structure comprises a stent. In some embodiments, the stent comprises a drug eluting stent. In some embodiments, the method comprises applying a rotating magnetic field along an axis concentric with the rod thereby translating the second curved body toward the first curved body. In some embodiments, the first curved body, the second curved body, or a combination thereof, comprise a hemisphere shape. In some embodiments, the one or more frequencies comprise one or more resonant frequencies of the blood vessel plaque. In some embodiments, the alternating magnetic field comprises an alternating magnetic field in a first dimension, a second dimension, a third dimension, or any combination thereof. In some embodiments, the first dimension, the second dimension, and the third dimension are orthogonal dimensions with respect to each other. In some embodiments, the curved body is a sphere. In some embodiments, a third body is coupled to the curved body or the first curved body, the second curved body, or a combination thereof. In some embodiments, the third body comprises a coil body or a helical body. In some embodiments, the third body extends orthogonally away from a surface of the curved body or the first curved body, the second curved body, or a combination thereof. In some embodiments, the third body is coupled to the curved body or the first curved body, the second curved body, or a combination thereof, at an apex of a curved cross-section of the curved body or a curved cross-section of the first curved body, a curved cross-section of the second curved body, or a combination thereof. In some embodiments, the third body maintains an orientation of the curved body or the first curved body, the second curved body, or a combination thereof, in the blood vessel of the subject with respect to the alternating magnetic field. In some embodiments, the third body comprises a length of up to about 15 millimeters. In some embodiments, the blood vessel plaque comprises a calcified plaque, a fibrofatty plaque, a fibrous plaque, a mixed plaque, or a combination of any one or more thereof. In some embodiments, the alternating magnetic field is provided by an electromagnet. In some embodiments, the alternating magnetic field is provided by one or more coils surrounding the blood vessel of the subject. In some embodiments, the fractured blood vessel plaque increases compliance of the blood vessel. In some embodiments, the curved body is injected into the blood vessel of the subject. In some embodiments, the method comprises obtaining or receiving data of the blood vessel plaque volume, morphology, location, or any combination thereof, prior to, or after fracturing the blood vessel plaque. In some embodiments, the method comprises providing a magnetic field to translate or transport the curved body from a first location in the blood vessel of the subject to a second location in the blood vessel of the subject, wherein the first location and the second location do not spatially overlap. In some embodiments, the first curved body is spaced at a distance of up to about 15 millimeters (mm) from the second curved body.

Aspects of the disclosure describe an intravascular device, comprising: a first curved body coupled to a second curved body; one or more disks disposed between the first curved body and the second curved body; and at least two cutting elements disposed on the one or more disks, wherein a first cutting element of the at least two cutting elements is coupled to a first magnet, wherein a second cutting element of the at least two cutting elements is coupled to a second magnet, and wherein the first cutting element and the second cutting element extend outward from an outer surface of the one or more disks when a magnetic field is provided to the first magnet, the second magnet, or a combination of one or more thereof. In some embodiments, the first cutting element, the second cutting element, or a combination thereof, is rotatably coupled to a disk of the one or more disks. In some embodiments, the first cutting element is coupled to a disk of the one or more disks via a first disk mounting interface, and wherein the second cutting element is coupled to the disk of the one or more disks via a second disk mounting interface. In some embodiments, the first cutting element comprises a first curved surface that contacts a second curved surface of the second cutting element, and wherein the second cutting element extends outward from the outer surface of the one or more disks when the first curved surface of the first cutting element applies a force to the second curved surface of the second cutting element. In some embodiments, the device further comprises a magnet disposed within the first curved body, the second curved body, or a combination thereof. In some embodiments, the one or more disks comprise a third cutting element. In some embodiments, the third cutting element is coupled to a third magnet, and wherein the third cutting element extends outward from the outer surface of the one or more disks when the magnetic field is provided to the first magnet, the second magnet, the third magnet, or a combination thereof. In some embodiments, the third cutting element comprises a third curved surface in contact with the first curved surface of the first cutting element, the second curved surface of the second cutting element, or a combination thereof, and wherein the third cutting element extends outward from the outer surface of the one or more disks when the first curved surface of the first cutting element, the second curved surface of the second cutting element, or a combination thereof, apply a force to the third curved surface of the third cutting element. In some embodiments, a first disk of the one or more disks comprises a first orientation of the first cutting element, the second cutting element, the third cutting element, or a combination thereof, and wherein a second disk of the one or more disks comprises a second orientation of a fourth cutting element and a fifth cutting element. In some embodiments, the first orientation of the first cutting element and the second cutting element comprises a staggered position of the first cutting element and the second cutting element with respect to a position of the fourth cutting element and the fifth cutting element in the second orientation. In some embodiments, the first orientation of the first cutting element and the second cutting element comprises a first position of the first cutting element and a second position of the second cutting element that are up to about 45 degrees or up to about 90 degrees offset from a fourth position of the fourth cutting element and fifth position of the fifth cutting element in the second orientation. In some embodiments, the first curved body, the second curved body, or a combination thereof, comprise a hemisphere shaped curved body. In some embodiments, the device comprises a third body coupled to the first curved body, the second curved body, or a combination thereof. In some embodiments, the third body comprises a helical body, a cylindrical body, a coil body, or any combination thereof. In some embodiments, the third body extends orthogonally away from a surface of the first curved body, the second curved body, or a combination thereof. In some embodiments, the surface comprises a surface orthogonal to a surface of the first curved body, a surface orthogonal to a surface of the second curved body, or a combination thereof. In some embodiments, the third body comprises a length of at least about a diameter of the first curved body or at least about a diameter of the second curved body. In some embodiments, the magnetic field is provided by an electromagnet. In some embodiments, the magnetic field is provided along an axis concentric with the first curved body, the second curved body, or a combination thereof. In some embodiments, the magnetic field rotates about an axis concentric with the first curved body, the second curved body, or a combination thereof. In some embodiments, the first curved body, the second curved body, or a combination thereof comprise a diameter of up to about 3 micrometers (μm) or up to about 4 μm. In some embodiments, the first curved body, the second curved body, the one or more disks disposed between the first curved body and the second curved body, or any combination thereof, is not coupled to a guidewire. In some embodiments, the first curved body is spaced a distance of up to about five times a diameter of the first curved body or up to about five times a diameter of the second curved body, from the second curved body.

Aspects of the disclosure describe a method of treating or alleviating a blood vessel plaque, comprising: alternating a magnetic field about an axis concentric with a first curved body coupled to a second curved body disposed in a blood vessel of a subject thereby rotating a first cutting element, a second cutting element, or a combination thereof, wherein the first cutting element, the second cutting element, or a combination thereof, are disposed on one or more disks coupled to the first curved body and the second curved body, wherein the first cutting element, the second cutting element, or a combination thereof, contact a surface of an inner lumen of the blood vessel adjacent to a blood vessel plaque to treat or alleviate the blood vessel plaque of a subject, and wherein the alternating magnetic field is provided perpendicular to the axis of the first curved body, the axis of the second curved body, or a combination thereof, external to the blood vessel. In some embodiments, the blood vessel plaque comprises a calcified plaque, a fibrofatty plaque, a fibrous plaque, a mixed plaque, or a combination of any one or more thereof. In some embodiments, the blood vessel plaque comprises an artery or arteriole plaque. In some embodiments, the first cutting element comprises a first magnet, and wherein the second cutting element comprises a second magnet. In some embodiments, the method comprises providing a constant magnetic field to the first magnet of the first cutting element and the second magnet of the second cutting element thereby extending the first cutting element, the second cutting element, or a combination thereof, away from an outer curved surface of the one or more disks. In some embodiments, the first cutting element, the second cutting element, or a combination thereof, is rotatably coupled to a disk of the one or more disks. In some embodiments, the first cutting element is coupled to a disk of the one or more disks via a first disk mounting interface, and wherein the second cutting element is coupled to the disk of the one or more disks via a second disk mounting interface. In some embodiments, the first cutting element comprises a first curved surface that contacts a second curved surface of the second cutting element, and wherein the second cutting element extends outward from the outer curved surface of the one or more disks when the first curved surface of the first cutting element applies a force to the second curved surface of the second cutting element. In some embodiments, the method further comprises a fourth magnet disposed within the first curved body, the second curved body, or a combination thereof. In some embodiments, the one or more disks comprises a third cutting element. In some embodiments, the third cutting element is coupled to a third magnet, and wherein the third cutting element extends outward from the outer curved surface of the one or more disks when the magnetic field is provided to the first magnet, the second magnet, the third magnet or a combination thereof. In some embodiments, the third cutting element comprises a third curved surface in contact with the first curved surface of the first cutting element, the second curved surface of the second cutting element, or a combination thereof, and wherein the third cutting element extends outward from the outer curved surface of the one or more disks when the first curved surface of the first cutting element, the second curved surface of the second cutting element, or a combination thereof, apply a force to the third curved surface of the third cutting element. In some embodiments, a first disk of the one or more disks comprises a first orientation of the first cutting element and the second cutting element, and wherein a second disk of the one or more disks comprises a second orientation of a fourth cutting element and a fifth cutting element. In some embodiments, the first orientation of the first cutting element and the second cutting element comprises a staggered position of the first cutting element and the second cutting element with respect to a position of the fourth cutting element and the fifth cutting element in the second orientation. In some embodiments, the first orientation of the first cutting element and the second cutting element comprises a first position of the first cutting element and a second position of the second cutting element that are up to about 45 degrees or up to about 90 degrees offset from a fourth position of the fourth cutting element and fifth position of the fifth cutting element in the second orientation. In some embodiments, the first curved body, the second curved body, or a combination thereof, comprise a hemisphere shaped curved body. In some embodiments, a third body is coupled to the first curved body, the second curved body, or a combination thereof. In some embodiments, the third body comprises a helical body, a cylindrical body, a coil body, or a combination thereof. In some embodiments, the third body extends orthogonally away from a surface of the first curved body, the second curved body, or a combination thereof. In some embodiments, the surface comprises a surface orthogonal to a surface of the first curved body, a surface orthogonal to a surface of the second curved body, or a combination thereof. In some embodiments, the third body comprises a length of up to about 15 mm. In some embodiments, the magnetic field is provided by an electromagnet. In some embodiments, the method further comprises injecting the first curved body coupled to the second curved body into the blood vessel of the subject. In some embodiments, the method further comprises implanting the first curved body coupled to the second curved body into the blood vessel of the subject. In some embodiments, the method further comprises retrieving the first curved body coupled to the second curved body from the blood vessel of the subject. In some embodiments, the first curved body coupled to the second curved body is retrieved from the blood vessel of the subject with a magnetic filter. In some embodiments, the first curved body, the second curved body, or a combination thereof comprise a diameter of up to about 3 micrometers (μm) or up to about 4 um. In some embodiments, the first curved body, the second curved body, the one or more disks, or any combination thereof, are not coupled to a guidewire. In some embodiments, the first curved body is spaced at a distance of up to about five times a diameter of the first curved body or up to about five times a diameter of the second curved body, from the second curved body.

Aspects of the disclosure describe a method of treating or alleviating a blood vessel plaque, comprising: providing a plurality of atherectomy devices within a blood vessel of a subject, wherein each atherectomy device of the plurality of atherectomy devices comprises a first curved body coupled to a second curved body, wherein the first curved body, the second curved body, or a combination thereof are coupled to one or more disks, and wherein a first cutting element, a second cutting element, or a combination thereof, are disposed on the one or more disks; and alternating a magnetic field about an axis concentric with the plurality of atherectomy devices thereby rotating the first cutting element, the second cutting element, or a combination thereof, to treat or alleviate the blood vessel plaque of the subject, wherein the first cutting element, the second cutting element, or a combination thereof, contact a surface of an inner lumen of the blood vessel adjacent to a blood vessel plaque, and wherein the alternating magnetic field is provided perpendicular to the axis concentric with the plurality of atherectomy devices external to the blood vessel. In some embodiments, the blood vessel plaque comprises a calcified plaque, a fibrofatty plaque, a fibrous plaque, a mixed plaque, or a combination of any one or more thereof. In some embodiments, the blood vessel plaque comprises an artery or arteriole plaque. In some embodiments, the first cutting element comprises a first magnet, and wherein the second cutting element comprises a second magnet. In some embodiments, the method further comprises providing a constant magnetic field to the first magnet of the first cutting element and the second magnet of the second cutting element thereby extending the first cutting element, the second cutting element, or a combination thereof, away from an outer curved surface of the one or more disks. In some embodiments, the first cutting element, the second cutting element, or a combination thereof, is rotatably coupled to a disk of the one or more disks. In some embodiments, the first cutting element is coupled to a disk of the one or more disks via a first disk mounting interface, and wherein the second cutting element is coupled to the disk of the one or more disks via a second disk mounting interface. In some embodiments, the first cutting element comprises a first curved surface that contacts a second curved surface of the second cutting element, and wherein the second cutting element extends outward from the outer curved surface of the one or more disks when the first curved surface of the first cutting element applies a force to the second curved surface of the second cutting element. In some embodiments, a fourth magnet is disposed within the first curved body, the second curved body, or a combination thereof. In some embodiments, the one or more disks comprises a third cutting element. In some embodiments, the third cutting element is coupled to a third magnet, and wherein the third cutting element extends outward from the outer curved surface of the one or more disks when the magnetic field is provided to the first magnet, the second magnet, the third magnet or a combination thereof. In some embodiments, the third cutting element comprises a third curved surface in contact with the first curved surface of the first cutting element, the second curved surface of the second cutting element, or a combination thereof, and wherein the third cutting element extends outward from the outer curved surface of the one or more disks when the first curved surface of the first cutting element, the second curved surface of the second cutting element, or a combination thereof, apply a force to the third curved surface of the third cutting element. In some embodiments, a first disk of the one or more disks comprises a first orientation of the first cutting element and the second cutting element, and wherein a second disk of the one or more disks comprises a second orientation of a fourth cutting element and a fifth cutting element. In some embodiments, the first orientation of the first cutting element and the second cutting element comprises a staggered position of the first cutting element and the second cutting element with respect to a position of the fourth cutting element and the fifth cutting element in the second orientation. In some embodiments, the first orientation of the first cutting element and the second cutting element comprises a first position of the first cutting element and a second position of the second cutting element that are up to about 45 degrees or up to about 90 degrees offset from a fourth position of the fourth cutting element and fifth position of the fifth cutting element in the second orientation. In some embodiments, the first curved body, the second curved body, or a combination thereof, comprise a hemisphere shaped curved body. In some embodiments, a third body is coupled to the first curved body, the second curved body, or a combination thereof. In some embodiments, the third body comprises a helical body, a cylindrical body, a coil body, or a combination thereof. In some embodiments, the third body extends orthogonally away from a surface of the first curved body, the second curved body, or a combination thereof. In some embodiments, the surface comprises a surface orthogonal to a surface of the first curved body, a surface orthogonal to a surface of the second curved body, or a combination thereof. In some embodiments, the third body comprises a length of up to about 15 mm. In some embodiments, the magnetic field is provided by an electromagnet. In some embodiments, the method further comprises injecting the plurality of atherectomy devices into the blood vessel of the subject. In some embodiments, the method further comprises implanting the plurality of atherectomy devices into the blood vessel of the subject. In some embodiments, the method further comprises retrieving the plurality of atherectomy devices from the blood vessel of the subject. In some embodiments, the plurality of atherectomy devices is retrieved with a magnetic filter. In some embodiments, the first curved body, the second curved body, or a combination thereof comprise a diameter of up to about 3 micrometers (μm) or up to about 4 um. In some embodiments, the first curved body, the second curved body, the one or more disks, or any combination thereof, are not coupled to a guidewire. In some embodiments, the first curved body is spaced at a distance of up to about five times a diameter of the first curved body or up to about five times a diameter of the second curved body, from the second curved body.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C show an isometric (FIG. 1A), side view (FIG. 1B), and cross-sectional view (FIG. 1C), of an angioplasty intravascular device, as described in some embodiments herein;

FIGS. 2A-2C show an isometric (FIG. 2A), side view (FIG. 2B), and cross-sectional view (FIG. 2C), of an angioplasty intravascular device deploying and/or expanding a stent, as described in some embodiments herein;

FIGS. 3A-3B shown an isometric (FIG. 3A), and side view (FIG. 3B) of a stent, as described in some embodiments herein;

FIGS. 4A-4D show various steps of a method of using an intravascular device to fracture intravascular plaque with an external magnetic field (FIGS. 4A-4B), deploying and/or expanding a stent (FIG. 4C), and removal of the intravascular device once the stent has been deployed and/or expanded (FIG. 4D), as described in some embodiments herein;

FIGS. 5A-5G show an isometric (FIG. 5A), side view (FIG. 5B), cross-sectional view (FIG. 5C), a back perspective isometric view (FIG. 5D), a side view of a third body (FIG. 5E), and a cross-sectional view of the third body (FIG. 5F) of an intravascular device, and a fluid flow diagram resulting from the rotation of the third body (FIG. 5G), as described in some embodiments herein;

FIGS. 6A-6D show a top (FIGS. 6A and 6C) and isometric (FIGS. 6B and 6D) view of an expanded (FIGS. 6C and 6D) and retracted (FIGS. 6A and 6B) four element cutting disk, as described in some embodiments herein;

FIGS. 7A-7F show a top perspective view of a retracted (FIGS. 7A, 7C, and 7E), and expanded (FIGS. 7B, 7D, and 7F) three element cutting disk, as described in some embodiments herein;

FIGS. 8A-8C show an isometric (FIG. 8A), top view (FIG. 8B), and side view (FIG. 8C) of an intravascular atherectomy unit of the intravascular atherectomy device, as described in some embodiments herein;

FIGS. 9A-9C show an isometric (FIG. 9A), top view (FIG. 9B), and side view (FIG. 9C) of a curved body intravascular atherectomy device, as described in some embodiments herein;

FIGS. 10A-10D show an isometric (FIG. 10A), top view (FIG. 10B), side view (FIG. 10C), and back isometric perspective view (FIG. 10D) of a curved body stacked disk intravascular atherectomy device, as described in some embodiments herein;

FIG. 11 shows the various states of the retracted and expanded atherectomy device within a blood vessel to treat blood vessel plaque and corresponding polarity of applied magnetic field, as described in some embodiments herein;

FIGS. 12A-12C show orientations of a magnetic field generator and the angioplasty and/or the atherectomy intravascular device to induce motion of translation (FIG. 12A), rotation (FIG. 12B), and vibration (FIG. 12C);

FIG. 13 shows a system with a processor to interact with and/or control the various devices and sub system components, as described in some embodiments herein;

FIG. 14 shows a flow diagram of a method of fracturing, shattering, and/or breaking a blood vessel plaque, as described in some embodiments herein;

FIG. 15 shows a flow diagram of a method of alleviating and/or treating a blood vessel plaque, as described in some embodiments herein; and

FIG. 16 shows a flow diagram of a method of alleviating and/or treating a blood vessel plaque with a swarm of intravascular devices, as described in some embodiments herein.

### DETAILED DESCRIPTION OF THE INVENTION

The devices, system, and/or methods, described elsewhere herein, may be used to conduct one or more angioplasty and/or an atherectomy procedure(s) in a subject. In some cases, the angioplasty procedure may comprise fracturing, shattering, and/or breaking a blood vessel plaque and/or changing the mechanical structure of the blood vessel plaque of a subject. In some instances, the angioplasty procedure may comprise expanding an intravascular stent to increase an inner diameter of a blood vessel of the subject. In some cases, the atherectomy procedure may comprise altering, shaving, cutting, and/or removing a blood vessel plaque and/or other intravascular tissue to increase an inner diameter of the blood vessel of the subject. In some cases, the one or more angioplasty procedure(s) may be conducted with an angioplasty intravascular device, described elsewhere herein. In some instances, the one or more atherectomy procedure(s) may be conducted with an atherectomy intravascular device, described elsewhere herein.

In some cases, the angioplasty and/or atherectomy intravascular device(s) may be injected, provided, and/or delivered into a blood vessel of a subject for the angioplasty and/or atherectomy intravascular device to perform corresponding angioplasty and/or atherectomy procedures, described elsewhere herein. In some instances, the angioplasty and/or atherectomy intravascular device(s) may be implanted in the subject. In some cases, the devices, systems, and/or methods, described elsewhere herein, may translate, rotate, vibrate, expand, and/or collapse through an interaction of one or more magnets disposed within an angioplasty and/or atherectomy device, and one or more magnetic fields provided by a magnetic field generator external to the blood vessel. In some cases, the magnetic field generator may be coupled to one or more mechanical arms. In some instances, the one or more mechanical arms may comprise one or more robotic arms. In some instances, the magnetic field generator may comprise an electromagnet. In some cases, the magnetic field provided and/or outputted by the magnetic field generator may be alternated in polarity and/or pulsed using a controller. In some cases, the controller may comprise a microcontroller. In some cases, the angioplasty and/or atherectomy intravascular device(s) may not be coupled to a guide wire and/or catheter to translate, rotate, expand, vibrate, and/or collapse the angioplasty and/or atherectomy intravascular device(s). In some instances, the devices, systems, and/or methods described herein may be used to conduct an angioplasty and/or atherectomy procedure in a subject without the use of a guidewire and/or catheter coupled to the angioplasty and/or atherectomy device(s).

### Angioplasty Intravascular Device

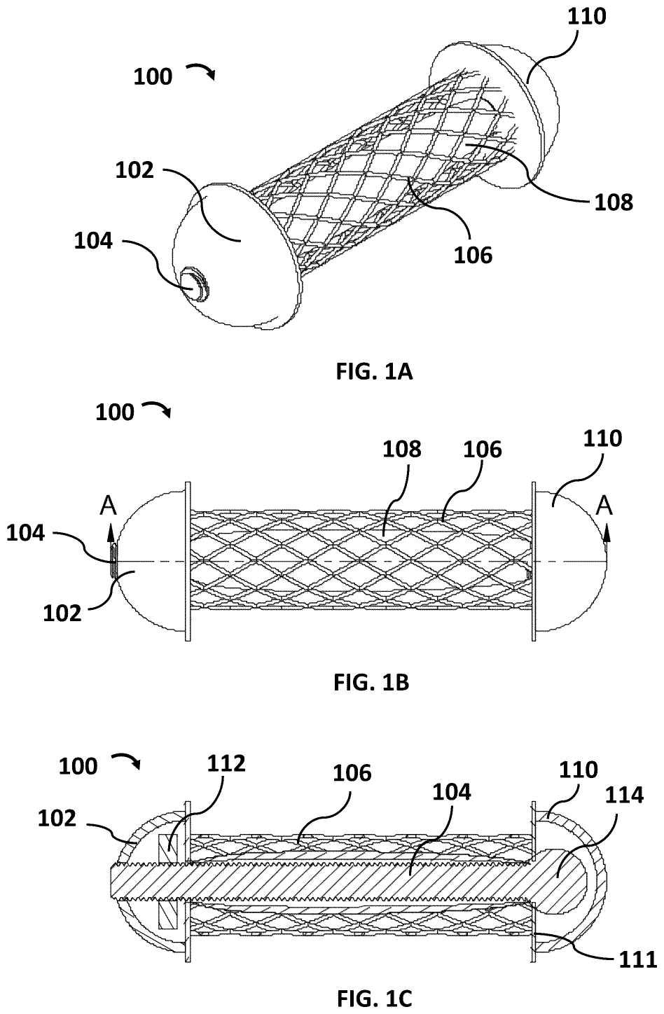

Aspects of the disclosure describe an angioplasty intravascular device (100, 200), as shown in FIGS. 1A-1C, 2A-2C, 3A-3B, 4A-4D and/or 5A-5G In some cases, the angioplasty intravascular device 100 may comprise: a first curved body 110 coupled to a rod 104 at a first end of the rod, where a magnet 114 is disposed within the first curved body 110; a mesh structure 106 concentric with the rod 104, where the mesh structure 106 comprises an inner surface and an outer surface; a deformable body 108 adjacent to the inner surface of the mesh structure 106; and a second curved body 102 coupled to a second end of the rod 104. In some cases, the second curved body 102 may apply a force to the deformable body 108 thereby expanding the mesh structure 106 when the second curved body 102 is translated toward the first curved body 110 at the first end of the rod 104. In some cases, the first curved body, the second curved body, or a combination of one or more thereof, may comprise a hemisphere curved body. In some cases, the magnet 114 may be coupled to the rod 104 at the first end of the rod. In some cases, the magnet 114 may be coupled to an inner structure 111 of the first curved body 110. In some cases, the inner structure 111 of the first curved body 110 may comprise a washer and/or a bearing. In some cases, the inner structure 111 may maintain a spatial position of the magnet 114 and rod 104 when the magnet 114 and rod 104 are rotated along a central axis of the rod 104. In some cases, the first curved body 110, the second curved body 102, or a combination of one or more thereof, may comprise a flat surface. In some instances, the flat surface of the first curved body 110, the flat surface of the second curved body 102, or a combination of one or more thereof, may contact an external surface of the deformable body 108. In some cases, the first curved body 110, the second curved body 102, or a combination of one or more thereof, may be made of a biocompatible material. The biocompatible material may comprise a biocompatible plastic, silicon, or a combination of one or more thereof. In some cases, the first curved body 110, the second curved body 102, the rod 104, the mesh structure 106, the deformable body 108, or any combination of one or more thereof, may not be coupled to a guide wire. In some cases, the first curved body 110 may be spaced a distance of up to about 15 millimeters (mm) from the second curved body 102.

In some cases, the magnet 114 may comprise a first pole and a second pole, where the first pole is orthogonal to the second pole. In some cases, the first pole, the second pole, or a combination of one or more thereof may be orthogonal to a central axis of the rod 104. In some instances, the magnet 114 may be coupled to the rod 104, where the magnet 114, the rod 104, or a combination of one or more thereof, may rotate within the first curved body 110. In some instances, the magnet 114 coupled to the rod 104 may be concentric with the inner structure 111 of the first curved body 110.

The deformable body 108 may encapsulate a fluid. In some instances, the fluid encapsulated by the deformable body 108 may comprise a density, buoyancy, and/or osmolarity of about an osmolarity, density, and/or buoyancy of mammalian blood. In some cases, the deformable body 108 may comprise a balloon. In some cases, the deformable body 108 may comprise a hole through a length of the deformable body 108. In some instances, the rod 104 may be concentric with, disposed within, and/or provided within the hole of the deformable body 108. In some instances, the deformable body 108 may be made of heat set material, plastic, or a combination of one or more thereof. In some cases, the deformable body 108 may be made of a thermoplastic polymer. In some cases, the deformable body 108 may be made of polyethylene terephthalate (PET), nylon, polyethylene (e.g., polyethylene with additives), polyvinyl chloride (PVC), polyurethane, or any combination of one or more thereof. In some instances, the deformable body 108 may comprise a material that changes from a first diameter to a second diameter when the second curved body 102 applies a force to the deformable body 108, where the first diameter is less than the second diameter. In some cases, the deformable body 108 may change from the second diameter to the first diameter when the second curved body 102 is translated away from the first curved body 110.

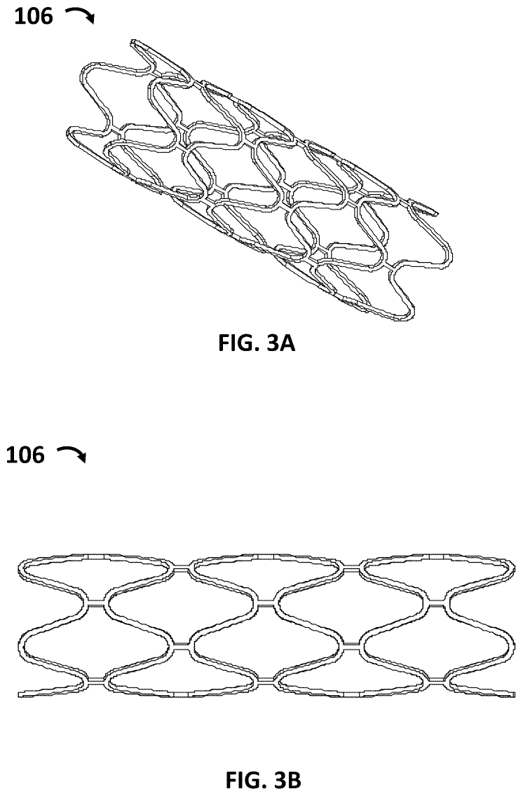

In some cases, the mesh structure 106 may comprise a stent, as shown in FIGS. 3A-3B. In some cases, the mesh structure 106 may comprise a drug-eluting stent, e.g., a dual drug-eluting stent (DDES), a gene eluting stent, a stem-cell carrying stent, or a radioactive stent. In some instances, the mesh structure 106 may comprise a material of a biodegradable mesh. In some instances, the material of the mesh structure 106 may comprise zinc, magnesium, polymer, or any combination of one or more thereof. In some cases, the mesh structure 106 may be made of a biocompatible metal, e.g., nitinol.

In some instances, the rod 104 may comprise a threaded rod. In some cases, the threaded rod 104 may be coupled to the first curved body 110, the second curved body 102, or a combination of one or more thereof. In some cases, the second curved body 102 may comprise an opening, where the opening of the second curved body is concentric with the rod 104. In some instances, the opening may comprise a threaded surface reversibly mated to the threaded rod. In some cases, the second curved body 102 may comprise a threaded mounting interface 112 disposed within the second curved body 102. In some cases, the threaded mounting interface 112 may comprise a nut. In some cases, the threaded mounting interface 112 may comprise an opening concentric and/or cordial with the opening of the second curved body 102, the rod 104, or a combination of one or more thereof. In some instances, the opening of the threaded mounting interface may comprise a threaded surface reversibly mated to the threaded rod.

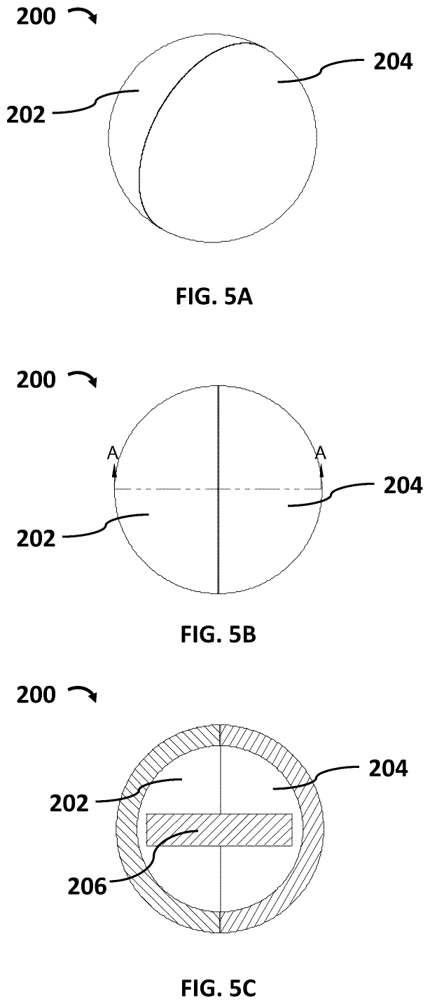

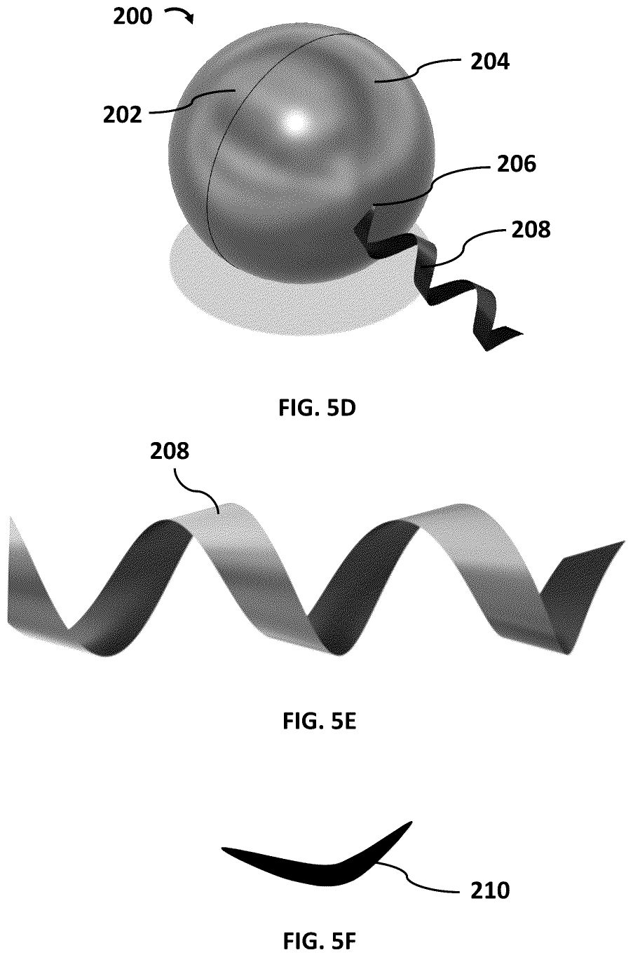

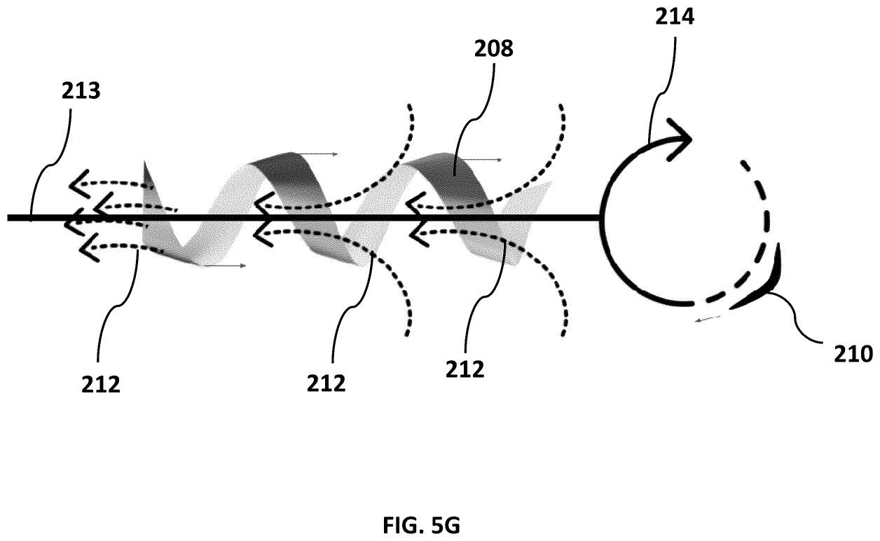

In some cases, the angioplasty intravascular device 200, as shown in FIGS. 5A-5G, may comprise: a first curved body 202 coupled to a second curved body 204; a magnet 206 disposed within the first curved body 202, the second curved body 204, or a combination of one or more thereof; and a third body 208 coupled to the first curved body 202, the second curved body 204, or a combination of one or more thereof, where the third body 208 extends orthogonally away from a surface of the first curved body 202, a surface of the second curved body 204, or a combination of one or more thereof, and where the third body 208 comprises a length of about a diameter of the first curved body 202 coupled to the second curved body 204. In some instances, the first curved body 202, the second curved body 204, or a combination of one or more thereof, may comprise a hemisphere curved body. In some cases, the first curved body 202, the second curved body 204, or a combination of one or more thereof, may be hollow. In some cases, the first curved body 202, the second curved body 204, or a combination of one or more thereof may made of a biocompatible material. In some cases, the biocompatible material may comprise a biocompatible polymer. In some cases, the first curved body, the second curved body, or a combination of one or more thereof, may be made of metal. In some cases, the surface of the first curved body 202, the surface of the second curved body 204, or a combination of one or more thereof may comprise a surface orthogonal to the first curved body 202, a surface orthogonal to the second curved body 204, or a combination of one or more thereof. In some cases, the angioplasty intravascular device 200, may not be coupled to a guide wire. In some instances, the magnet 206 may be fixed within the first curved body 202, the second curved body 204, or a combination of one or more thereof.

In some cases, the angioplasty intravascular device(s) (100, 200) may comprise a third body 208 coupled to the first curved body (110, 202), second curved body (102, 204), or a combination of one or more thereof. In some cases, the third body 208 may extend orthogonally away from a surface of the first curved body (110, 202), second curved body (102, 204), or a combination of one or more thereof. In some instances, the third body 208 may comprise a tail of the first curved body (110, 202), a tail of the second curved body (102, 204), or a combination of one or more thereof. In some cases, the third body 208 may be coupled to the first curved body (110, 202), second curved body (102, 204), or a combination of one or more thereof, at an apex of a curved surface of the first curved body (110, 202), an apex of a curved surface of the second curved body (102, 204), or a combination of one or more thereof. In some instances, the third body 208 may comprise a helical body. In some cases, the third body 208 may comprise a rigid body. In some cases, the third body 208 may comprise a cylindrical shape. In some instances, the third body 208 may comprise a spiral and/or helical shape. In some cases, the third body 208 may comprise a length of about a length from an end of the first curved body (110, 202) to an end of the second curved body (102, 204). In some cases, the third body 208 may comprise a length of about a diameter of the first curved body (110, 202) or a length of about a diameter of the second curved body (102, 204). In some instances, the third body 208 may comprise a diameter of up to about 25% of a diameter of the first curved body (110, 202) or a diameter of up to about 25% of a diameter of the second curved body (102, 204). In some cases, when the third body 208 coupled to the first curved body (110, 220), second curved body (102, 204), or a combination of one or more thereof, is rotated, the third body 208 may apply a force (e.g., a pushing force 212) to the first curved body (110, 202), the second curved body (102, 204), or a combination of one or more thereof, thereby translating the first curved body (110, 202), the second curved body (102, 204), or a combination of one or more thereof. In some instances, the third body 208 may comprise a propeller coupled to the first curved body (110, 202), the second curved body (102, 204), or a combination of one or more thereof. In some cases, the third body 208 may comprise a curved cross-section 210, as shown in FIG. 5F. In some cases, the curved cross-section 210 may comprise a shallow crescent shape. In some cases, the curved cross-section 210 of the third body 208 when rotated 214 may propel fluid 212 external and/or adjacent to an outer surface of the third body towards a central axis 213 of the third body 208 and out an end of the third body 208, as shown in FIG. 5G. In some cases, the third body 208 may comprise a counterclockwise spiral orientation. In some instances, the counterclockwise spiral orientation of the third body 208 when rotated counterclockwise may propel and/or provide a pushing force 212 to the first curved body (110, 202), the second curved body (102, 204), or a combination of one or more thereof, thereby translating the first curved body (110, 202), the second curved body (102, 204), or a combination of one or more thereof. In some cases, the third body 208 may comprise a clockwise spiral orientation. In some instances, the clockwise spiral orientation of the third body 208 when rotated clockwise may propel and/or provide a pushing force 212 to the first curved body (110, 202), the second curved body (102, 204), or a combination of one or more thereof, thereby translating the first curved body (110, 202), the second curved body (102, 204), or a combination of one or more thereof.

### Atherectomy Intravascular Device

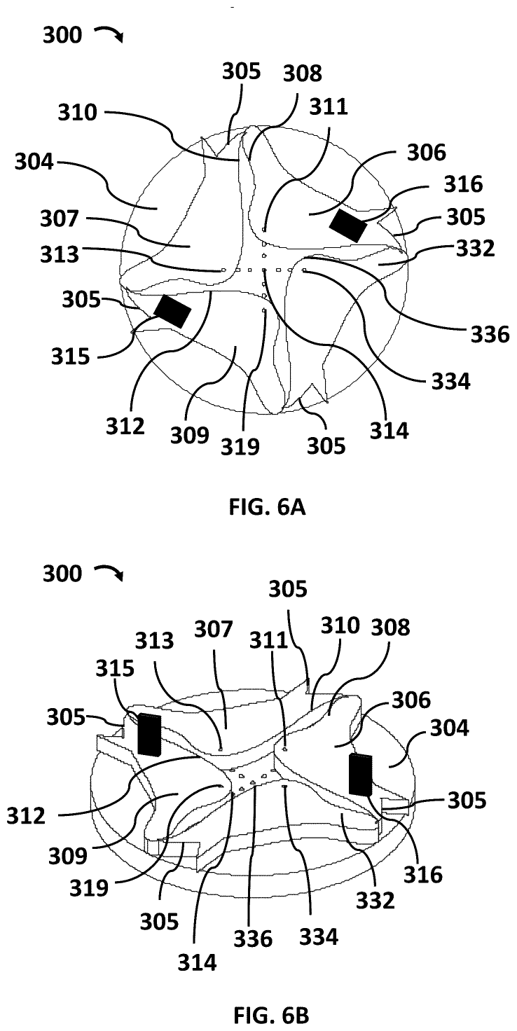

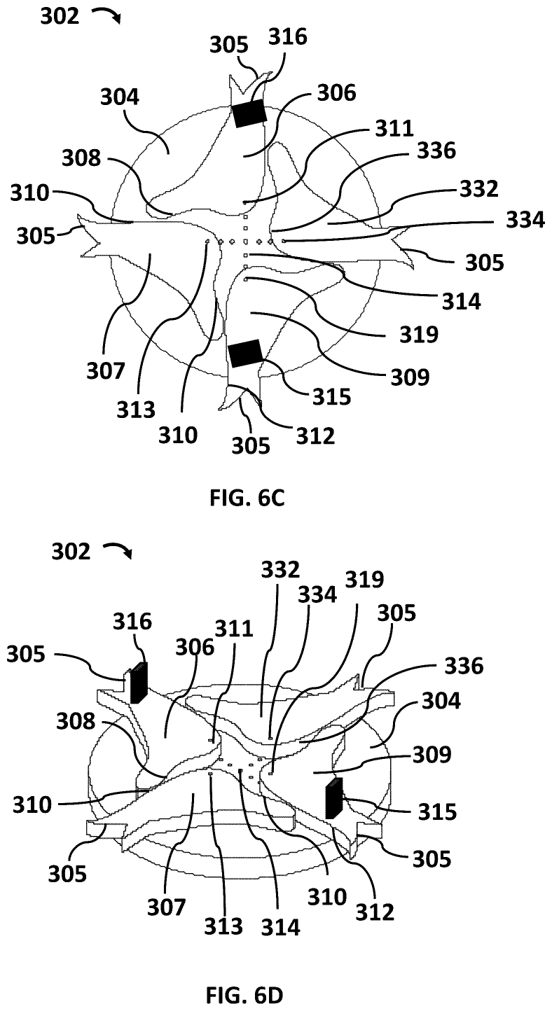

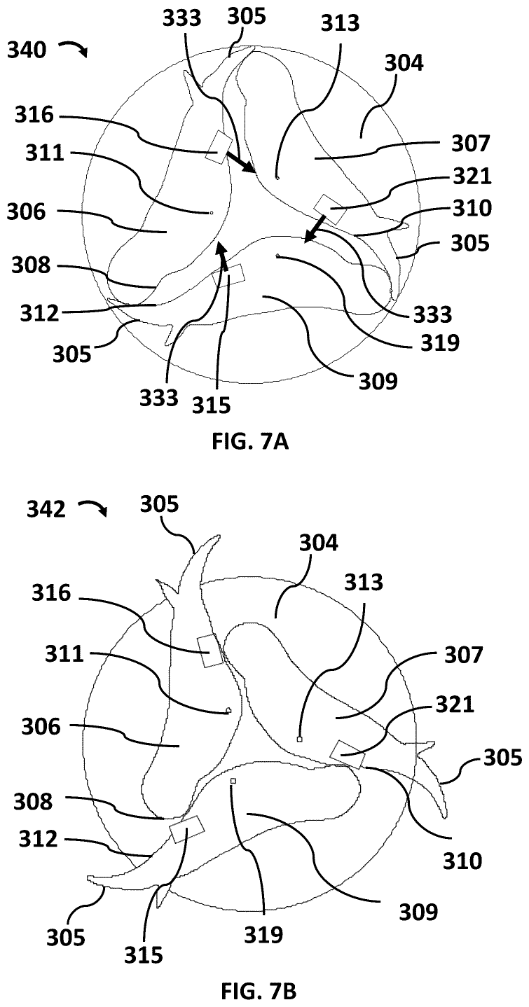

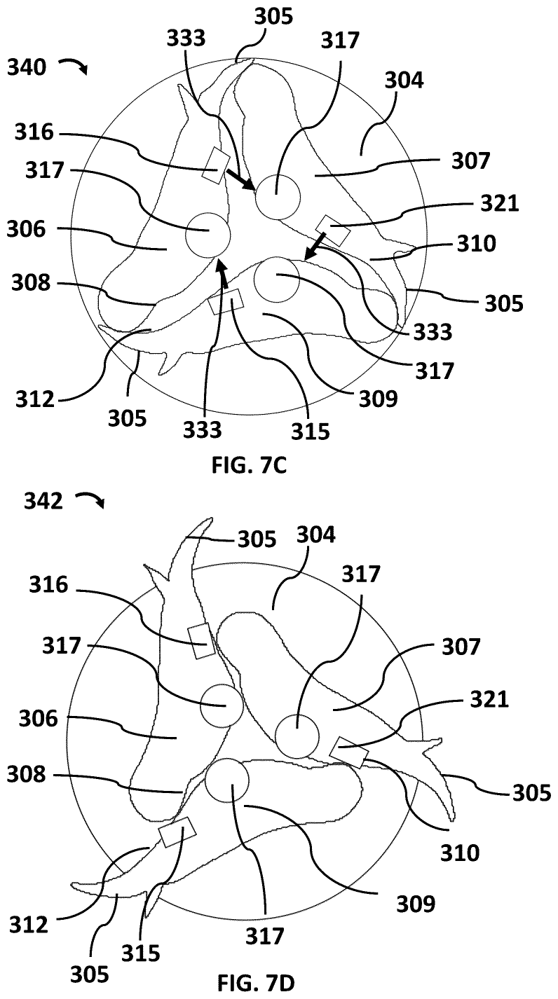

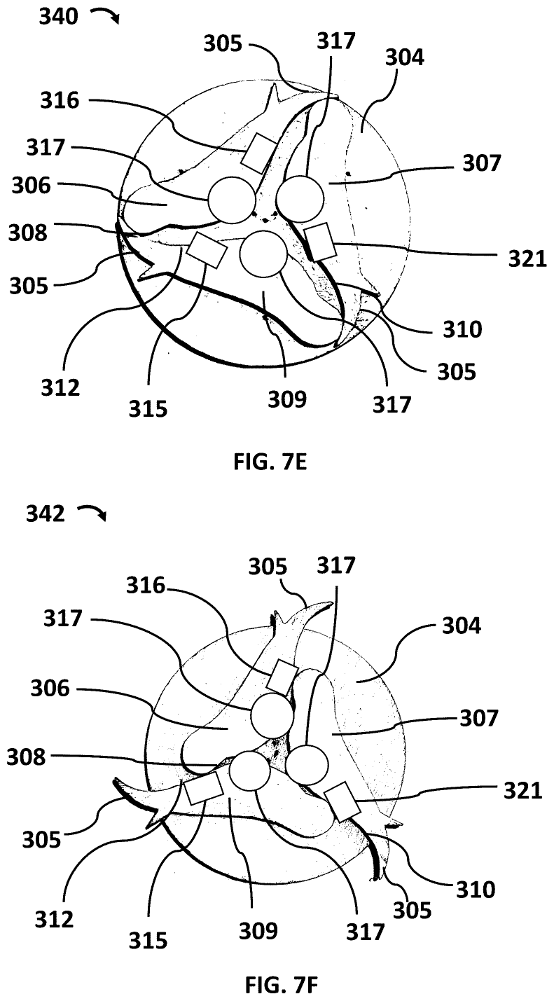

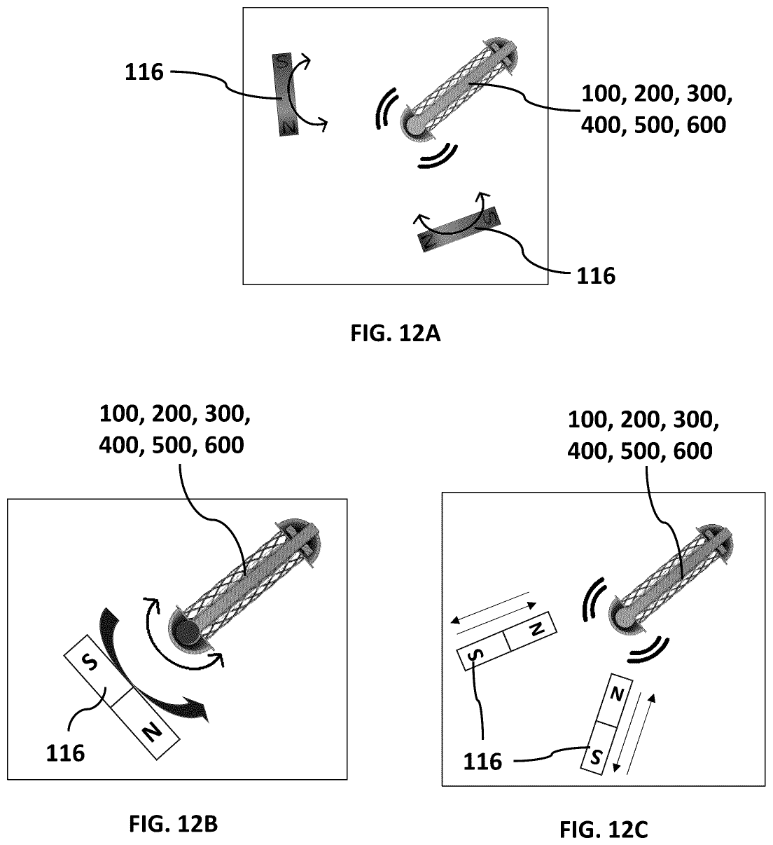

In some embodiments, the atherectomy intravascular device (300, 302, 340, 342, 400, 500, 600), as shown in FIGS. 6A-6D, 7A-7F, 8A-8C, 9A-9C, and 10A-10D, may comprise: a first body 320 coupled to a second body 322; one or more disks (304, 324, 326) disposed between the first body 320 and the second body 322; and at least two cutting elements (306, 307, 309, 328, 330, 332) disposed on the one or more disks (304, 324, 326), where a first cutting element 306 of the at least two cutting elements is coupled to a first magnet 316, where a second cutting element 309 of the at least two cutting elements is coupled to a second magnet 315, and where the first cutting element 306 and the second cutting element 309 extend outward from an outer surface of the one or more disks (304, 324, 326) when a magnetic field is provided to the first magnet 316, the second magnet 315, or a combination of one or more thereof. In some cases, the at least two cutting elements (306, 307, 309, 328, 330, 332), may comprise a cutting surface 305. In some cases, the first body 320, the second body 322, or a combination of one or more thereof, may comprise a diameter of up to about 3 micrometers (μm), or up to about 4 μm. In some cases, the first body 320, the second body 322, or a combination of one or more thereof, may comprise a diameter of up to about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 3.9 μm, or up to about 4.0 μm. In some instances, the first cutting element 306, the second cutting element 309, or a combination of one or more thereof, may be rotatably coupled to a disk of the one or more disks (304, 324, 326). In some instances, a magnet may be disposed within the first body 320, the second body 322, or a combination of one or more thereof. In some instances, the first body 320 and the second body 322 may comprise curved bodies. In some cases, the one or more disks (304, 324, 326) may comprise a third cutting element 307 and/or a fourth cutting element 332. In some cases, the third cutting element 307 may comprise a third magnet 321, as shown in FIGS. 7A-7F. In some cases, a north or south pole of the first magnet 316, the second magnet 315, the third magnet 321, or a combination of one or more thereof, may be oriented along a direction 333 away from an outer surface of the one or more disks towards a central region of the one or more disks (304, 324, 326), as shown in FIG. 7A. In some cases, the orientation of the pole of the first magnet 316, the second magnet 315, the third magnet 321, or a combination of one or more thereof, may provide a benefit of extending the one or more cutting elements (306, 307, 309, 328, 330, 332) disposed on the one or more disks (304, 324, 326) in any orientation of magnetic field provided to the intravascular atherectomy device external to the blood vessel by the magnetic field generator. In some cases, the first curved body 320, the second curved body 322, or a combination of one or more thereof, may comprise a hemisphere shaped curved body. In some instances, the magnetic field may be provided by a magnetic field generator 116, described elsewhere herein. In some cases, the magnetic field generator 116 may be an electromagnet. In some cases, the magnetic field may be provided along an axis concentric with the first body 320, the second body 322, or a combination of one or more thereof. In some instances, the magnetic field 117 may rotate (e.g., as shown in FIG. 12B) about an axis concentric with the first body 320, the second body 322, or a combination of one or more thereof. In some cases, the first body 320, the second body 322, the one or more disks (304, 324, 326) disposed between the first body 320 and the second body 322, or any combination of one or more thereof, may not be coupled to a guidewire. In some cases, the first body 320 may be spaced a distance of at least about four times the diameter of the first body 320 or at least about four times the diameter the second body 322, from the second body 322. In some cases, the first body 320 may be spaced a distance of up to about five times the diameter of the first body 320 or up to about five times the diameter the second body 322, from the second body 322.

In some cases, a first north pole surface of the first magnet 316 may comprise a first surface normal vector 180 degrees from a second surface normal vector of a second north pole surface of the second magnet 315. In some instances, a first south pole surface of the first magnet 316 may comprise a first surface normal vector 180 degrees from a second surface normal vector of a second south pole surface of the second magnet 315.

In some instances, a third body 208 may be coupled to the first body 320 and/or the second body 322. In some cases, the third body 208 may comprise a helical body, a cylindrical body, spiral body, a coil body, or any combination of one or more thereof. In some instances, the third body 208 may extend orthogonally away from a surface of the first body 320, the second body 322, or a combination of one or more thereof. In some cases, the third body 208 may comprise a length of at least about a diameter of the first body 320 or at least about a diameter of the second body 322. In some cases, the third body 208 may comprise a length of up to about a distance between the first body 320 and the second body 322.

In some cases, the first cutting element 306 may couple to a disk of the one or more disks (304, 324, 326) via a first disk mounting interface 314. In some embodiments, the second cutting element 309 may couple to the disk of the one or more disks (304, 324, 326) via a second disk mounting interface 314. In some instances, the third cutting element 307 may couple to the disk of the one or more disks (304, 324, 326) via a third disk mounting interface 314. In some cases, the fourth cutting element 332 may couple to the disk of the one or more disks (304, 324, 326) via a fourth disk mounting interface 314. In some cases, the first mounting interface 314 may couple to the first cutting element 306 via a first cutting element mounting interface 311. In some cases, the second cutting element 309 may couple to the second disk mounting interface 314 via a second cutting element mounting interface 319. In some instances, the third cutting element 307 may couple to the third disk mounting interface 314 via a third cutting element mounting interface 313. In some cases, the fourth cutting element 332 may couple to the fourth disk mounting interface 314 via a fourth cutting element mounting interface 334. In some cases, the first cutting element 306, the second cutting element 309, the third cutting element 307, the fourth cutting element 332, or a combination of one or more thereof, may be pivotably coupled to the first, second, third, and/or fourth disk mounting interface(s) 314. In some cases, the first cutting element 306, the second cutting element 309, the third cutting element 307, the fourth cutting element 332, or a combination of one or more thereof, may be pivotably coupled to the first, second, third, and/or fourth disk mounting interfaces 314 with a pivot joint and/or pivot fastener 317, as shown in FIGS. 7C-7F. In some cases, the first cutting element 306 may comprise a first curved surface 308 that contacts a third curved surface 310 of a third cutting element 307 and/or a fourth curved surface 336 of the fourth cutting element 332, and where the third cutting element 307 and/or the fourth cutting element 332 may extend outward from the outer surface of the one or more disks (304, 324, 326) when the first curved surface 308 of the first cutting element 306 applies a force to the third curved surface 310 of the third cutting element 307 and/or the fourth curved surface 336 of the fourth cutting element 332, as seen in FIGS. 6A-6D. In some instances, the second cutting element 309 may be coupled to a second magnet 315, and where the second cutting element 309 may extend outward from the outer surface of the one or more disks (304, 324, 326) when a magnetic field 117 (e.g., provided by a magnetic field generator 116), described elsewhere herein, is provided to the first magnet 316, the second magnet 315, or a combination of one or more thereof. In some cases, the second cutting element 309 may comprises a second curved surface 312 in contact with the third curved surface 310 of the third cutting element 307 and/or the fourth curved surface 336 of the fourth cutting element 332. In some cases, the second cutting element 309 may extend outward from the outer surface of the one or more disks (304, 324, 326) when a magnetic field 117 provided by the magnetic field generator 116 is provided to the second magnet 315. In some cases, the third cutting element 307 and/or the fourth cutting element 332 may extend outward from the outer surface of the one or more disks (304, 324, 326) when the second curved surface 312 of the second cutting element 309 applies a force to the third curved surface 310 of the third cutting element 307 and/or when the second curved surface 312 of the second cutting element 309 applies a force to the fourth curved surface 336 of the fourth cutting element 332.

In some cases, the first cutting element 306 and/or the second cutting element 309 may extend outward from the outer surface of the one or more disks (304, 324, 326) when the third curved surface 310 of the third cutting element 307 and/or the fourth curved surface 336 of the fourth cutting element 332 apply a force to the first curved surface 308 of the first cutting element 306 and/or when the third curved surface 310 of the third cutting element 307 and/or the fourth curved surface 336 of the fourth cutting element 332 apply a force to the second curved surface 312 of the second cutting element 309.

In some cases, the first cutting element 306 may comprise a first curved surface 308 that may contact a second curved surface 312 of a second cutting element 309, and where the second cutting element 309 may extend outward from the outer surface of the one or more disks (304, 324, 326) when the first curved surface 308 of the first cutting element 306 applies a force to the second curved surface 312 of the second cutting element 309, as seen in FIGS. 7A-7F. In some cases, the third curved surface 310 of the third cutting element 307 may be in contact with the second curved surface 312 of the second cutting element 309 and/or the third curved surface 310 of the third cutting element 307 may be in contact with the first curved surface 308 of the first cutting element 306. In some cases, the third cutting element 307 may extend outward from the outer surface of the one or more disks (304, 324, 326), when the second curved surface 312 and/or the first curved surface 308 apply a force to the third curved surface 310. In some instances, the second cutting element 309 may be coupled to a second magnet 315, and where the second cutting element 309 may extend outward from the outer surface of the one or more disks (304, 324, 326) when a magnetic field 117 (e.g., provided by a magnetic field generator 116), described elsewhere herein, is provided to the first magnet 316, the second magnet 315, or a combination of one or more thereof. In some cases, the second cutting element 309 may comprises a second curved surface 312 in contact with the third curved surface 310 of the third cutting element 307 and/or the first curved surface 308 of the first cutting element 306, and where the second cutting element 309 may extend outward from the outer surface of the one or more disks (304, 324, 326) when the third curved surface 310 of the third cutting element 307 and/or the first curved surface 308 of the first cutting element 306, applies a force to the second curved surface 312 of the second cutting element 309.

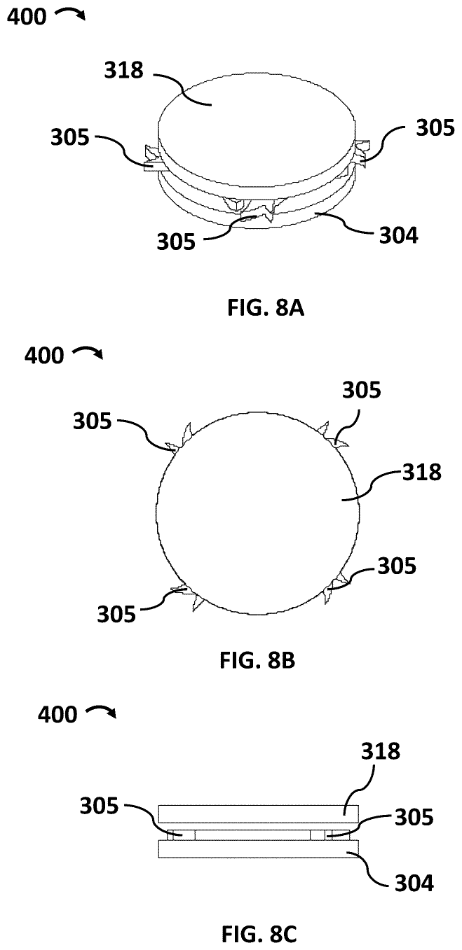

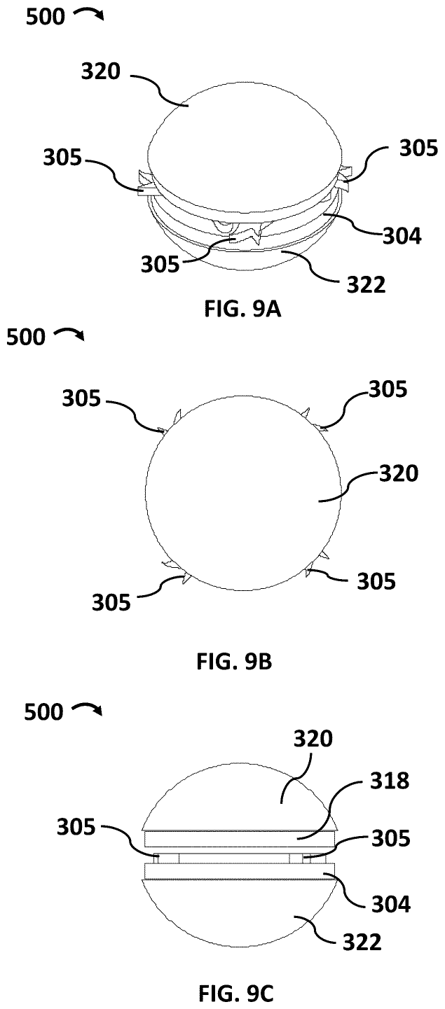

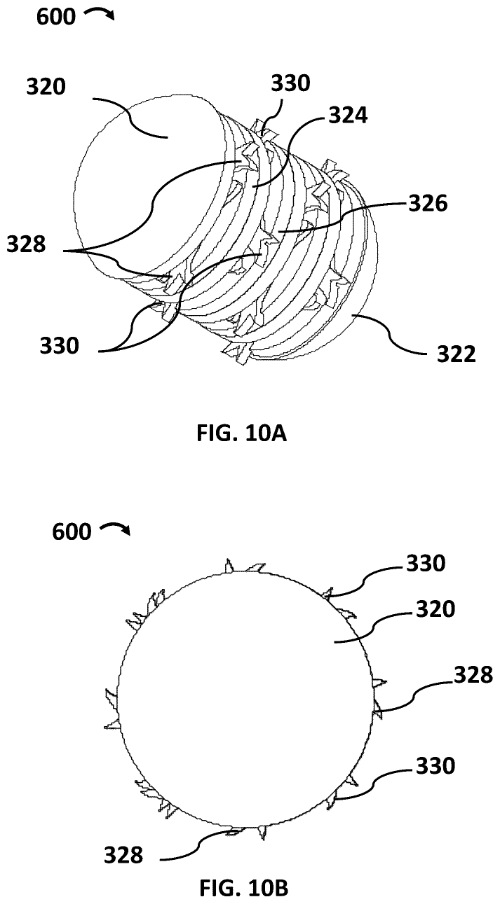

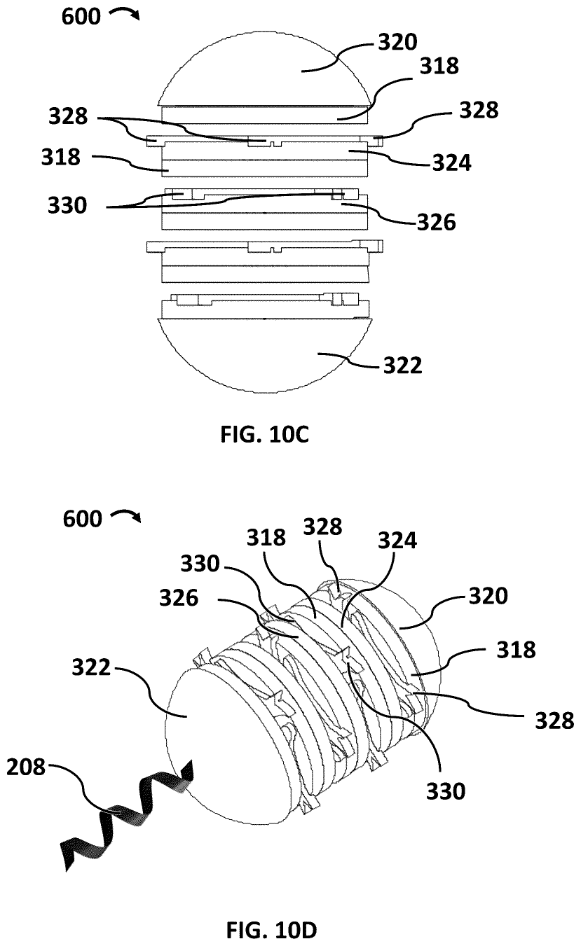

In some cases, a body 318 may be coupled to one or more cutting elements (306, 307, 309, 332) disposed on the one or more disks (304, 324, 326), as shown in FIGS. 8A-8C. In some cases, the body may be coupled to a surface of the one or more cutting elements (306, 307, 309, 332) parallel to the surface of the one or more disks (304, 324, 326) that the one or more cutting elements (306, 307, 309, 332) couple to. In some cases, the combined body 318 and one or more cutting elements (306, 307, 309, 330, 332) disposed on the one or more disks (304, 324, 326) may comprise a single atherectomy unit and/or atherectomy stack of a multi atherectomy unit and/or multi atherectomy stack atherectomy device, as shown in FIGS. 9A-9C and 10A-10D.

In some cases, a first disk 324 of the one or more disks (304, 324, 326) may comprise a first orientation 328 of the first cutting element, the second cutting element, the third cutting element, or a combination of one or more thereof, and where a second disk 326 of the one or more disks (304, 324, 326) may comprise a second orientation 330 of a fourth cutting element and/or a fifth cutting element. In some cases, the first orientation 328 of the first cutting element, the second cutting element, and/or the third cutting element may comprises a staggered position of the first cutting element, the second cutting element, and/or the third cutting element with respect to a position of the fourth cutting element and/or the fifth cutting element in the second orientation 330. In some instances, the staggered position may comprise a position of the first cutting element, the second cutting element, and/or the third cutting element, that is not adjacent to the fourth cutting element and/or the fifth cutting element. In some cases, the first orientation 328 of the first cutting element, the second cutting element, and/or the third cutting element, may comprise a first position of the first cutting element, a second position of the second cutting element, and/or a third position of the third cutting element, that are up to about 45 degrees or up to about 90 degrees offset from a fourth position of the fourth cutting element and/or a fifth position of the fifth cutting element in the second orientation 330. In some cases, the offset may comprise a radial offset along a circumference of the one or more disks (304, 324, 326).

### Methods

Provided herein are methods for treating, ameliorating, removing, fracturing, breaking, shaving, and/or reducing, one or more blood vessel plaques 120 of a subject. In some cases, the one or more blood vessel plaques 120 may comprise a calcified plaque, a fibrofatty plaque, a fibrous plaque, a mixed plaque, or a combination of any one or more thereof. In some cases, the blood vessel plaque 120 may comprise an artery plaque, an arteriole plaque, or a combination of one or more thereof.

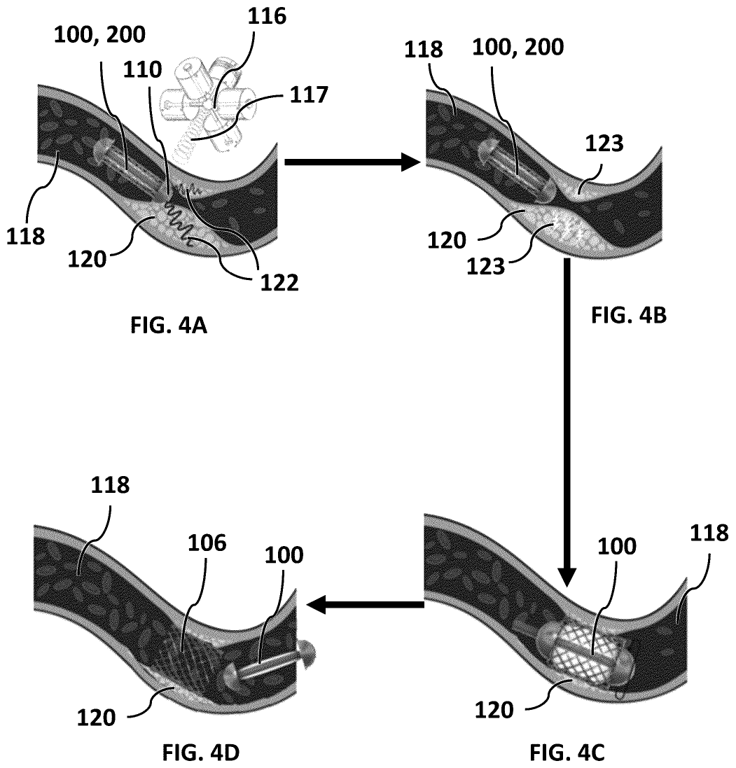

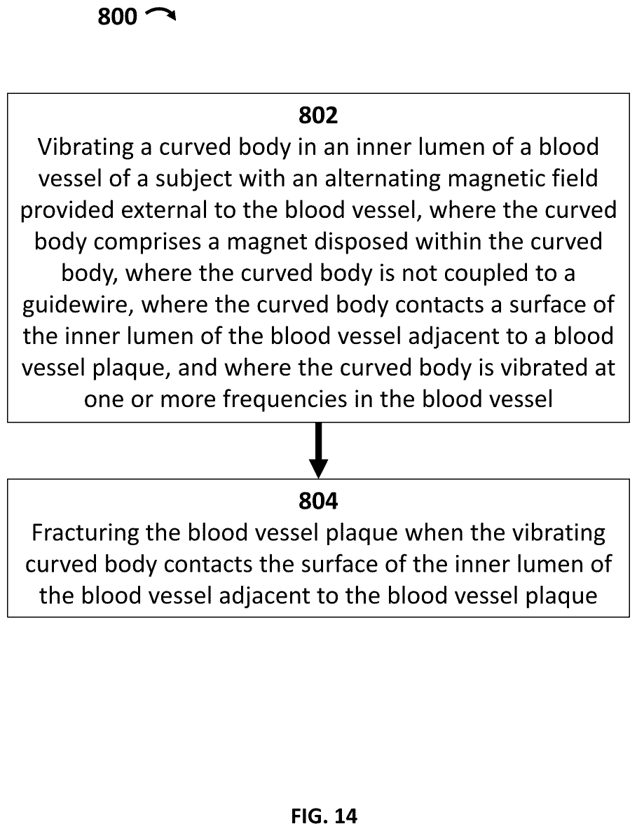

In some cases, the methods provided herein may comprise a method of fracturing an intravascular plaque 800, as shown in FIGS. 4A-4B and 14. In some cases, the method of fracturing an intravascular plaque 800, may comprise: vibrating a curved body (100, 200) in an inner lumen of a blood vessel 118 of a subject with an alternating magnetic field 117 (e.g., as shown in FIG. 12C) provided external to the blood vessel, where the curved body (100, 200) comprises a magnet (114, 206) disposed within the curved body, where the curved body is not coupled to a guidewire, where the curved body contacts a surface of the inner lumen of the blood vessel adjacent to a blood vessel plaque 120, and where the curved body is vibrated at one or more frequencies in the blood vessel 802; and fracturing the blood vessel plaque 123 when the vibrating curved body contacts the surface of the inner lumen of the blood vessel adjacent to the blood vessel plaque 804. In some instances, the method 800 may further comprise injecting the curved body (100, 200) into the blood vessel 118 of the subject. In some cases, one or more pressure waves 122 may be transmitted from the vibrating curved body (100, 200) to the blood vessel plaque 120 when the surface of the vibrating curved body contacts the surface of the inner lumen of the blood vessel adjacent to the blood vessel plaque 120, thereby fracturing and/or breaking 123 the blood vessel plaque 120. In some cases, the fractured blood vessel plaque 123 may increase compliance of the blood vessel, to, for example, allow the placement of a stent, described elsewhere herein. In some cases, the method 800 may comprise obtaining and/or receiving data of the blood vessel plaque 120 volume, morphology, location, mechanical properties, biochemical properties, or any combination of one or more thereof, prior to and/or after fracturing and/or breaking the blood vessel plaque 123. In some instances, the method 800 may further comprise providing a magnetic field to translate and/or to transport the curved body (100, 200) (as shown in FIG. 12A, described elsewhere herein) from a first location in the blood vessel 118 of the subject to a second location in the blood vessel of the subject, where the first location and the second location do not spatially overlap. In some cases, the magnetic field 117 provided by the magnetic field generator 116 to translate the curved body (100, 200) may be provided by one or more mechanical arms that are coupled to one or more magnetic field generators 116. In some instances, the one or more mechanical arms may comprise one or more robotic arms. In some cases, the one or more mechanical arms may translate the curved body (100, 200) by oscillating the spatial orientation of a north and a south polarity of a magnetic field generator 116 oriented perpendicular to the orientation of the north and south polarity of the magnet (114, 206) disposed in and/or coupled to the intravascular device (100, 200), and translating the one or more mechanical arms with the spatially oscillating magnetic field from a position adjacent to the intravascular device (100, 200) to a position a distance away from the intravascular device, where the distance away from the intravascular device is greater than a distance between the intravascular device (100, 200) and a distance to a position adjacent to the intravascular device (100, 200). In some cases, a first mechanical arm and a first magnetic field generator may be positioned at a first distance from the intravascular device, and where a second mechanical arm and a second magnetic field generator may be positioned at a second distance from the intravascular device, where the first distance may be less than the second distance.

In some cases, the curved body may comprise a first curved body (110, 202) coupled to a second curved body (102, 204), as described elsewhere herein. In some cases, the first curved body (110, 202), the second curved body (102, 204), or a combination of one or more thereof, may comprise a hemisphere shape. In some cases, the curved body (100, 200) may comprise a sphere. In some instances, the one or more frequencies may comprise one or more resonant frequencies of the blood vessel plaque 120. In some cases, the first curved body (110, 202) may be spaced at a distance of up to about 15 mm from the second curved body (102, 204). In some cases, the first curved body (110, 202) may be spaced at a distance of up to about 5 mm, about 10 mm, about 11 mm, about 12 mm, about 13 mm, about 14 mm, or about 15 mm from the second curved body (102, 204).

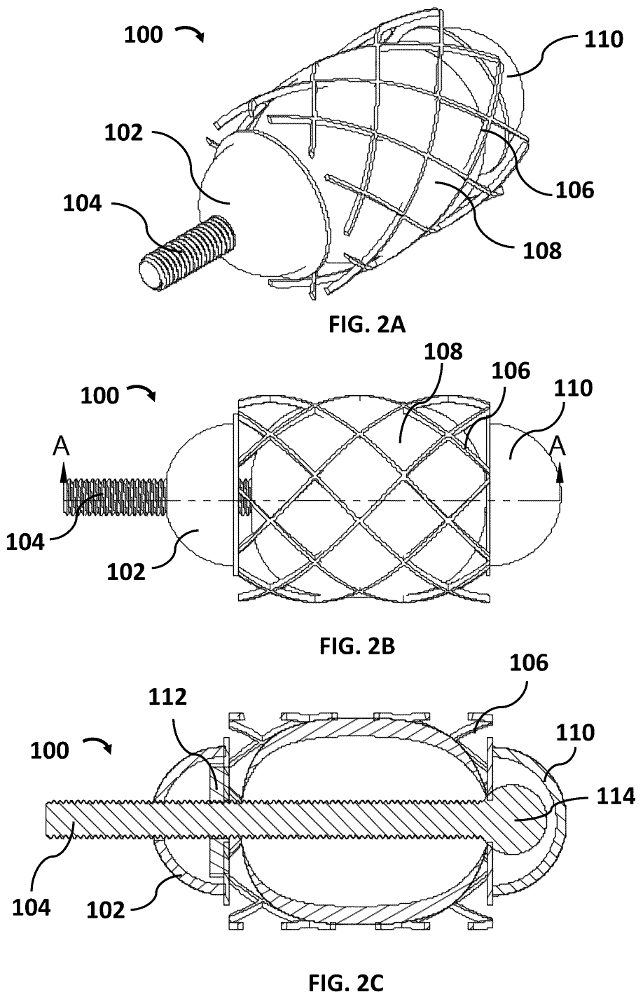

In some instances, the first curved body 110 and the second curved body 102 may be coupled to a rod 104. In some cases, the first curved body 110 may be disposed at a first end of the rod 104, and where the second curved body 102 may be disposed at a second end of the rod 104. In some instances, the rod 104 may comprise a threaded region on a surface of the rod. In some cases, the first curved body 110, the second curved body 102, or a combination of one or more thereof, may comprise a curved opening concentric with the rod 104. In some cases, the surface of the curved opening of the second curved body 102 may comprise a threaded region 112 reversibly mated to the threaded region of the surface of the rod 104. In some cases, the rod 104 may be concentric with the first curved body 110, the second curved body 102, or a combination of one or more thereof. In some instances, a surface of the rod 104 may be in contact with a surface of a deformable body 108, described elsewhere herein. In some instances, the first curved body 110, the second curved body 102, or a combination of one or more thereof, may comprise a flat surface. In some cases, the flat surface of the first curved body 110, the flat surface of the second curved body 102, or a combination of one or more thereof, may be in contact with an outer surface of the deformable body 108. In some cases, the method may further comprise applying a rotating magnetic field (e.g., as shown in FIG. 12B) along an axis concentric with the rod 104 thereby translating the second curved body 102 toward the first curved body 110, as shown in FIGS. 2A-2C.

In some cases, an outer surface of the deformable body 108 may be in contact with a mesh structure 106, and where the mesh structure 106 may be concentric with the rod 104. In some instances, the mesh structure 106 may comprise a stent.

In some cases, after one or more of fracturing and/or breaking of the blood vessel plaque 120, described elsewhere herein, a mesh structure 106 may be deployed, expanded, and/or placed at a region of the blood vessel plaque 120 to increase an inner diameter of the blood vessel as shown in FIGS. 4C-4D. In some instances, the stent may comprise a drug-eluting stent. In some cases, a rotational magnetic field 117 from a magnetic field generator 116 may be provided to magnet 114 disposed in the first curved body 110 (as shown in FIG. 12B) to rotate the magnet 114 and rod 104 coupled to the magnet 114. Upon rotation of the magnet 114 and rod, the second curved body 102 coupled to the rod 104 may translate toward the first curved body 110 contacting and applying a force to the deformable body 108. The deformable body 108 may then expand from a first diameter to a second diameter, where the first diameter is less than the second diameter, thereby applying a force to expand the mesh structure 106 in contact with an outer surface of the deformable body, described elsewhere herein. The mesh structure 106 may then be positioned over one or more regions of blood vessel plaque 120 to increase the inner diameter of the blood vessel lumen, as shown in FIG. 4C. Once the mesh structure 106 is deployed and/or expanded, the magnetic field generator 116 may adjust the rotation of the rotational magnetic field 117 from clockwise to counter-clockwise or counter-clockwise to clockwise to translate the second curved body 102 coupled to the rod 104 away from the first curved body 110 thereby reducing a diameter of the deformable body 108 from the second larger diameter to a smaller first diameter, described elsewhere herein. The curved body 100 comprising the first curved body 110, second curved body 102, rod 104, and deformable body 108, may then be translated away from the region of the blood vessel where the mesh structure was deployed, expanded, and/or placed, as shown in FIG. 4D.

In some cases, the alternating magnetic field 117 may be generated by a magnetic field generator 116, described elsewhere herein. In some cases, the alternating magnetic field may comprise an alternating magnetic field in a first dimension, a second dimensions, a third dimension, or any combination of one or more thereof. In some instances, the first dimension, the second dimension, and/or the third dimension may be orthogonal dimensions with respect to each other. In some cases, the alternating magnetic field 117 may be provided by an electromagnet. In some instances, the alternating magnetic field 117 may be provided by one or more coils surrounding the blood vessel of the subject.

In some instances, a third body 208, described elsewhere herein, may be coupled to the curved body (100, 200) and/or the first curved body (110, 202), the second curved body (102, 204), or a combination of one or more thereof. In some cases, the third body 208 may comprise a coil body or a helical body. In some instances, the third body 208 may extend orthogonally away from a surface of the curved body (100, 200), and/or the first curved body (110, 202), the second curved body (102, 204), or a combination of one or more thereof. In some cases, the third body 208 may be coupled to the curved body (100, 200), and/or the first curved body (110, 202), the second curved body (102, 204), or a combination of one or more thereof, at an apex of a curved cross-section of the curved body (100, 200), and/or an apex of a curved cross-section of the first curved body (110, 202), an apex of a curved cross-section of the second curved body (102, 204), or a combination of one or more thereof. In some instances, the third body may comprise a length of up to about 15 mm. In some cases, the third body 208 may maintain an orientation of the curved body (100, 200) and/or the first curved body (110, 202), the second curved body (102, 204), or a combination of one or more thereof, in a blood vessel of the subject 118 with respect to the alternating magnetic field 117. In some cases, the third body 208 may maintain the orientation of the curved body (100, 200) and/or the first curved body (110, 202), the second curved body (102, 204) such that a first pole of the magnet (114, 206) is parallel to the alternating magnetic field 117 emitted by the magnetic field generator 116, as shown in FIG. 12A.

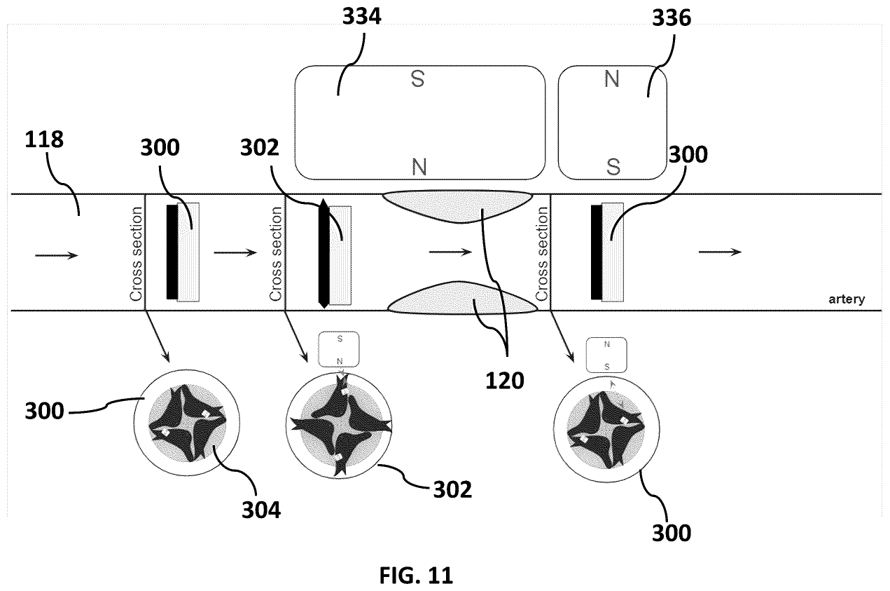

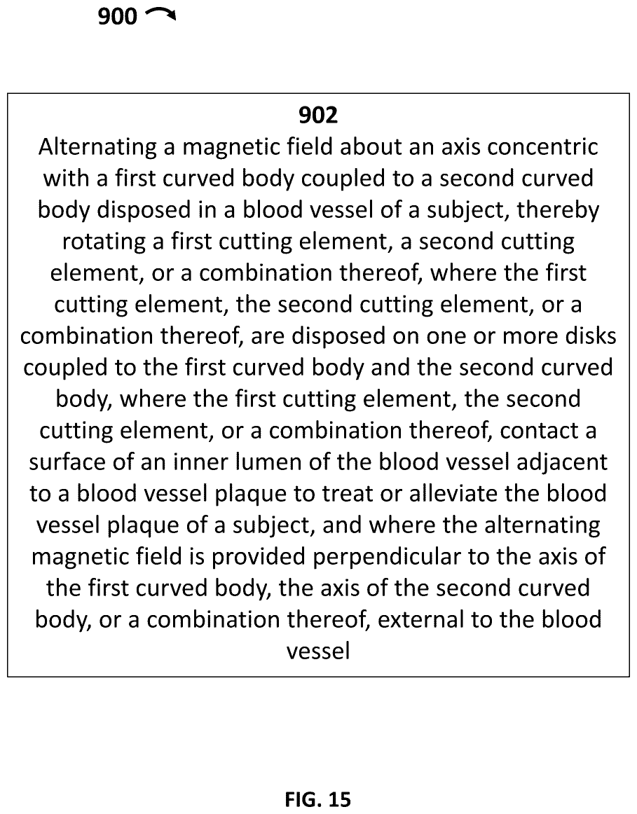

In some cases, the methods provided herein may comprise a method of treating and/or alleviating a blood vessel plaque 900, as shown in FIGS. 11 and 15. In some cases, the method of treating and/or alleviating a blood vessel plaque may comprise: alternating a magnetic field (e.g., as shown in FIG. 12B) about an axis concentric with a first curved body 320 coupled to a second curved body 322 disposed in a blood vessel of a subject, thereby rotating a first cutting element 306, a third cutting element 307, or a combination of one or more thereof, where the first cutting element 306, the third cutting element 307, or a combination of one or more thereof, are disposed on one or more disks (304, 324, 326) coupled to the first curved body 320 and the second curved body 322, where the first cutting element 306, the third cutting element 307, or a combination of one or more thereof, contact a surface of an inner lumen of the blood vessel adjacent to a blood vessel plaque 120 to treat or alleviate the blood vessel plaque of a subject, and where the alternating magnetic field is provided perpendicular to the axis of the first curved body 320, the axis of the second curved body 322, or a combination of one or more thereof, external to the blood vessel 902. In some cases, the blood vessel plaque 120 may comprise a calcified plaque, a fibrofatty plaque, a fibrous plaque, a mixed plaque, or a combination of any one or more thereof. In some cases, the blood vessel plaque 120 may comprise an artery plaque, an arteriole plaque, or a combination of one or more thereof. In some cases, the first curved body 320, the second curved body 322, or a combination of one or more thereof, may comprise a hemisphere shaped curved body. In some cases, the method 900 may comprise injecting the first curved body 320 coupled to the second curved body 322 into the blood vessel of the subject. In some instances, the method 900 may comprise implanting the first curved body 320 coupled to the second curved body 322 into the blood vessel of the subject. In some cases, the method 900 may comprise retrieving the first curved body 320 coupled to the second curved body 322 from the blood vessel of the subject. The first curved body 320 coupled to the second curved body 322 may be retrieved from the blood vessel of the subject with a magnetic filter. In some cases, the first curved body 320, the second curved body 322, or a combination of one or more thereof may comprise a diameter of up to about 3 μm or up to about 4 μm. In some cases, the first curved body 320, the second curved body 322, the one or more disks (304, 324, 326), or any combination of one or more thereof, may not be coupled to a guidewire. In some cases, the first curved body 320 may be spaced at a distance of up to about five times a diameter of the first curved body 320 or up to about five times a diameter of the second curved body 322, from the second curved body 322. In some cases, the first cutting element 306, the third cutting element 307, or a combination of one or more thereof, may be rotatably coupled to a disk of the one or more disks (304, 324, 326). In some instances, the magnetic field may be provided by an electromagnet.

In some instances, the first cutting element 306 may comprise a first magnet 316 and the third cutting element 307 may comprise comprises a third magnet 321, described elsewhere herein. In some cases, the method may further comprise providing a first magnetic field 334 to the first magnet 316 of the first cutting element and the third magnet 321 of the third cutting element 307 thereby extending the first cutting element, the third cutting element, the second cutting element, the fourth cutting element, or a combination of one or more thereof, away from an outer curved surface of the one or more disks 302, as shown in FIG. 11. In some cases, the method may comprise providing a second magnetic field 336 with an inverted polarity with respect to the first magnetic field 334 to the first magnet 316 of the first cutting element and the third magnet 321 of the third cutting element 307 to retract 300 the first cutting element, the third cutting element, the second cutting element, the fourth cutting element, or any combination of one or more thereof, as shown in FIG. 11. In some cases, the first magnetic field 334, the second magnetic field 336, or a combination of one or more thereof may be provided at spatially separate regions along a length of the blood vessel 118 of the subject, as shown in FIG. 11. In some cases, the first magnetic field 334 and/or the second magnetic field 336 may comprise a pulsed magnetic field and/or a continuous magnetic field. In some cases, the atherectomy device, described elsewhere herein, may be provided to the blood vessel of the subject in a retracted state 300, described elsewhere herein. A first magnetic field 334 may be provided to the atherectomy device when the atherectomy device is adjacent and/or about to come into contact with blood vessel plaque 120 such that the at least two cutting elements (306, 307, 309, 328, 330, 332), may be expanded 302 to interface with a surface of the blood vessel lumen and/or the blood vessel plaque 120 to treat and/or ameliorate the blood vessel plaque 120. After the atherectomy device translates and/or travels through the region of the blood vessel with plaque 120, the second magnetic field 336 may be provided to the atherectomy device to retract 300 the at least two cutting elements (306, 307, 309, 328, 330, 332), described elsewhere herein. In some cases, the first magnetic field 334 and/or the second magnetic field 336 may comprise alternating and/or constant magnetic fields, describe elsewhere herein.

In some instances, the first cutting element may be coupled to a disk of the one or more disks via a first disk mounting interface, and the second cutting element may be coupled to the disk of the one or more disks via a second disk mounting interface. In some cases, the first cutting element may comprise a first curved surface that contacts a second curved surface of the second cutting element, and where the second cutting element extends outward from the outer curved surface of the one or more disks when the first curved surface of the first cutting element applies a force to the second curved surface of the second cutting element.

In some cases, a fourth magnet may be disposed within the first curved body, the second curved body, or a combination of one or more thereof. In some instances, the one or more disks may comprise the third cutting element coupled to the third magnet, and where the third cutting element extends outward from the outer curved surface of the one or more disks when a magnetic field is provided to the first magnet, the second magnet, the third magnet, or any combination of one or more thereof. In some instances, the third cutting element may comprise a third curved surface in contact with the first curved surface of the first cutting element, the second curved surface of the second cutting element, or a combination of one or more thereof. In some cases, the third cutting element may extend outward from the outer curved surface of the one or more disks when the first curved surface of the first cutting element, the second curved surface of the second cutting element, or a combination of one or more thereof, apply a force to the third curved surface of the third cutting element.

In some cases, there may be varied orientations of the cutting elements on the one or more disks of the atherectomy device, including the cutting elements being offset or staggered between disks of the one or more disks. In some cases, a first disk of the one or more disks may comprise a first orientation of the first cutting element and the second cutting element, and where a second disk of the one or more disks may include a second orientation of a fourth cutting element and a fifth cutting element. In some instances, the first orientation of the first cutting element and the second cutting element may comprise a staggered position of the first cutting element and the second cutting element with respect to a position of the fourth cutting element and the fifth cutting element in the second orientation. In some cases, the first orientation of the first cutting element and the second cutting element may comprise a first position of the first cutting element and a second position of the second cutting element that are up to about 45 degrees or up to about 90 degrees offset from a fourth position of the fourth cutting element and fifth position of the fifth cutting element in the second orientation. In some cases, the offset may comprise a radial offset along a circumference of the one or more disks.

In some cases, a third body may be coupled to the first curved body, the second curved body, or a combination of one or more thereof. The third body may comprise a helical body, a cylindrical body, a coil body, or a combination of one or more thereof. In some instances, the third body may extend orthogonally away from a surface of the first curved body, the second curved body, or a combination of one or more thereof. In some instances, the surface may comprise a surface orthogonal to a surface of the first curved body, a surface orthogonal to a surface of the second curved body, or a combination of one or more thereof. In some cases, the third body may comprise a length of up to about 15 mm.

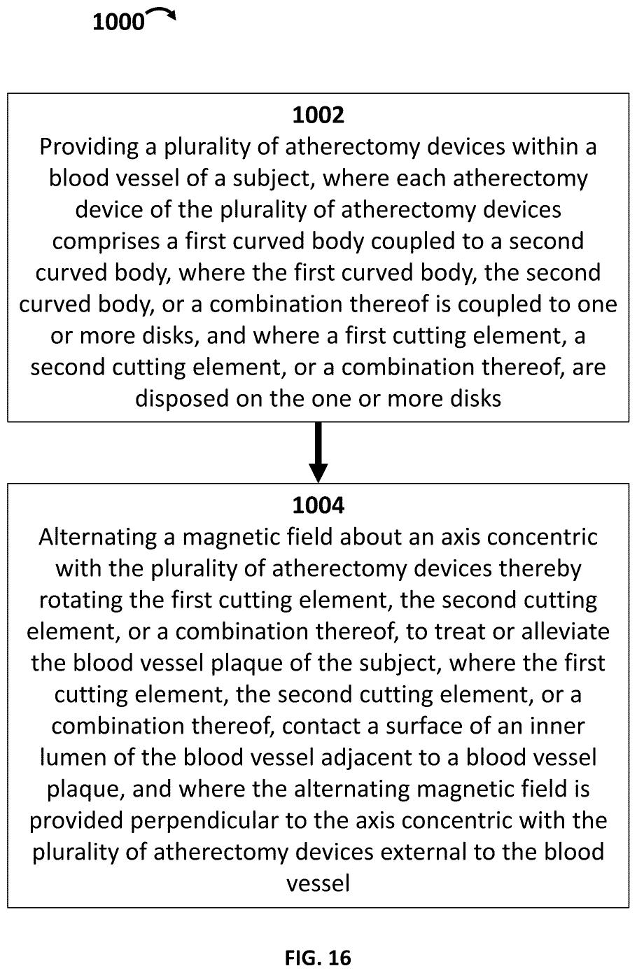

In some cases, the methods provided herein may comprise a method of treating and/or alleviating a blood vessel plaque 1000, as shown in FIG. 16. In some cases, the method 1000 may comprise: providing a plurality of atherectomy devices within a blood vessel of a subject, wherein each atherectomy device of the plurality of atherectomy devices comprises a first curved body coupled to a second curved body, where the first curved body, the second curved body, or a combination of one or more thereof is coupled to one or more disks, and where a first cutting element, a second cutting element, or a combination of one or more thereof, are disposed on the one or more disks 1002; and alternating a magnetic field about an axis concentric with the plurality of atherectomy devices thereby rotating the first cutting element, the second cutting element, or a combination of one or more thereof, to treat or alleviate the blood vessel plaque of the subject, where the first cutting element, the second cutting element, or a combination of one or more thereof, contact a surface of an inner lumen of the blood vessel adjacent to a blood vessel plaque, and where the alternating magnetic field is provided perpendicular to the axis concentric with the plurality of atherectomy devices external to the blood vessel 1004. In some cases, the first curved body, the second curved body, or a combination of one or more thereof, may comprise a hemisphere shaped curved body. In some cases, the blood vessel plaque may comprise a calcified plaque, a fibrofatty plaque, a fibrous plaque, a mixed plaque, or a combination of any one or more thereof. In some instances, the blood vessel plaque may comprise an artery plaque or arteriole plaque. In some cases, the first cutting element may comprise a first magnet, and the second cutting element may comprise a second magnet. In some cases, the method 1000 may further comprise providing a magnetic field to the first magnet of the first cutting element and the second magnet of the second cutting element, thereby extending the first cutting element, the second cutting element, or a combination of one or more thereof, away from an outer curved surface of the one or more disks. In some cases, the magnetic field may comprise a constant and/or pulsed magnetic field. In some cases, the magnetic field may be provided by an electromagnet. In some cases, the method 1000 may further comprise injecting the plurality of atherectomy devices into the blood vessel of the subject. In some instances, the method 1000 may further comprise implanting the plurality of atherectomy devices into the blood vessel of the subject. In some instances, the method 1000 may comprise retrieving the plurality of atherectomy devices from the blood vessel of the subject. In some cases, the plurality of atherectomy devices may be retrieved with a magnetic filter. In some cases, the first curved body, the second curved body, or a combination of one or more thereof may comprise a diameter of up to about 3 μm or up to about 4 μm. In some instances, the first curved body may be spaced at a distance of up to about five times a diameter of the first curved body or up to about five times a diameter of the second curved body, from the second curved body. In some cases, the first curved body, the second curved body, the one or more disks, or any combination of one or more thereof, may not be coupled to a guide wire.

In some cases, the first cutting element, the second cutting element, or a combination of one or more thereof, may be rotatably coupled to a disk of the one or more disks. In some instances, the first cutting element may be coupled to a disk of the one or more disks via a first disk mounting interface, and where the second cutting element may be coupled to the disk of the one or more disks via a second disk mounting interface. In some cases, the first cutting element may comprise a first curved surface that contacts a second curved surface of the second cutting element, and where the second cutting element extends outward from the outer curved surface of the one or more disks when the first curved surface of the first cutting element applies a force to the second curved surface of the second cutting element. In some cases, the one or more disks may comprise a third cutting element. In some instances, the third cutting element may be coupled to a third magnet, and where the third cutting element may extend outward from the outer curved surface of the one or more disks when the magnetic field is provided to the first magnet, the second magnet, the third magnet, or any combination of one or more thereof. In some cases, a fourth magnet may be disposed within the first curved body, the second curved body, or a combination of one or more thereof. In some cases, the third cutting element may comprise a third curved surface in contact with the first curved surface of the first cutting element, the second curved surface of the second cutting element, or a combination of one or more thereof, and where the third cutting element extends outward from the outer curved surface of the one or more disks when the first curved surface of the first cutting element, the second curved surface of the second cutting element, or a combination of one or more thereof, apply a force to the third curved surface of the third cutting element.

In some cases, a first disk of the one or more disks may comprise a first orientation of the first cutting element and the second cutting element, and where a second disk of the one or more disks may comprise a second orientation of a fourth cutting element and a fifth cutting element. In some instances, the first orientation of the first cutting element and the second cutting element may comprise a staggered position of the first cutting element and the second cutting element with respect to a position of the fourth cutting element and the fifth cutting element in the second orientation. In some cases, the first orientation of the first cutting element and the second cutting element may comprise a first position of the first cutting element and a second position of the second cutting element that are up to about 45 degrees or up to about 90 degrees offset from a fourth position of the fourth cutting element and fifth position of the fifth cutting element in the second orientation. In some instances, the offset may comprise a radial offset along a circumference of the one or more disks.

In some cases, a third body may be coupled to the first curved body, the second curved body, or a combination of one or more thereof. In some instances, the third body may comprise a helical body, a cylindrical body, a coil body, or a combination of one or more thereof. In some cases, the third body may extend orthogonally away from a surface of the first curved body, the second curved body, or a combination of one or more thereof. In some instances, the surface may comprise a surface orthogonal to a surface of the first curved body, a surface orthogonal to a surface of the second curved body, or a combination of one or more thereof. In some cases, the third body may comprise a length of up to about 15 mm.

### Systems

Provided herein are systems that may interface, control, and/or interact with one or more angioplasty intravascular devices, one or more atherectomy intravascular devices, or a combination of one or more thereof, described elsewhere herein. In some cases, the systems may implement one or more methods described elsewhere herein.

In some embodiments, the systems may comprise a system for vibrating an intravascular device, comprising: (a) curved body 100, where the curved body comprises: (i) a first curved body 110 coupled to a rod 104 at a first end of the rod, where a magnet 114 is disposed within the first curved body 110; (ii) a mesh structure 106 concentric with the rod 104, where the mesh structure 106 comprises an inner surface and an outer surface; (iii) a deformable body 108 adjacent to the inner surface of the mesh structure 106; and (iv) a second curved body 102 coupled to a second end of the rod 104, where the second curved body 102 is concentric with the rod 104; and (b) a magnetic field generator 116, where the magnetic field generator provides an alternating magnetic field 117 to the magnet 114 disposed within the first curved body 110, thereby vibrating the first curved body, the second curved body, or a combination of one or more thereof, where the curved body is provided to a blood vessel 118 of a subject, and where the magnetic field generator 116 is external to the blood vessel of the subject. In some cases, the rod 104 may comprise a threaded rod. In some instances, the second curved body 102 may comprise an opening, where the opening of the second curved body may be concentric with the rod 104. In some cases, the opening of the second curved body 102 may comprise a threaded surface reversibly mated to the threaded rod. In some instances, the second curved body 102 may comprise a threaded mounting interface 112 disposed within the second curved body 102, and where the threaded mounting interface 112 comprises an opening that may be concentric or cordial with the opening of the second curved body 102, the rod 104, or a combination of one or more thereof. In some cases, the first curved body 110, the second curved body 102, or a combination of one or more thereof, may comprise a hemisphere curved body. In some cases, the first curved body 110, the second curved body 102, the third body 208, or a combination of one or more thereof, may not be coupled to a guidewire. In some cases, the magnetic field generator 116 may be coupled to one or more mechanical arms. In some instances, the magnetic field generator may comprise a plurality of magnetic field generators. In some cases, the one or more mechanical arms may comprise one or more robotic arms. In some cases, a first mechanical arm may provide a first magnetic field of a first magnetic field generator to the intravascular device in the blood vessel, where a second mechanical arm may provide a second magnetic field of a second magnetic field generator to the intravascular device in the blood vessel, where the first magnetic field and the second magnetic field differ in the orientation of the magnetic field (as shown in FIGS. 12A-12C) with respect to the magnet (114, 206) of the intravascular device.

In some instances, the deformable body 108 may encapsulate a fluid. In some instances, the fluid may comprise an osmolarity and/or density of about an osmolarity of mammalian blood and/or about a density of mammalian blood. In some cases, the deformable body 108 may comprise a balloon. In some instances, the deformable body 108 may comprise a material that changes from a first diameter to a second diameter when the second curved body 102 applies a force to the deformable body 108, and where the first diameter is less than the second diameter. In some cases, the deformable body 108 may change from the second diameter to the first diameter when the second curved body 102 is translated away from the first curved body 110. In some cases, the material of the deformable body 108 may comprise a heat set material, plastic, or a combination of one or more thereof. In some instances, the mesh structure 106 may comprise a stent. In some cases, the stent may comprise a drug-eluting stent.

In some cases, the first curved body 110, the second curved body 102, or a combination of one or more thereof, may comprise a flat surface. In some instances, the flat surface of the first curved body, the flat surface of the second curved body, or a combination of one or more thereof, may contact an outer surface of the deformable body 108. In some cases, the first curved body 110, the second curved body 102, or a combination of one or more thereof, may be made of a biocompatible material. In some cases, the biocompatible material may comprise a biocompatible plastic or silicon material. In some instances, the first curved body 110 may be spaced a distance of up to about 15 millimeters from the second curved body 102.

In some embodiments, the systems may comprise a system for vibrating an intravascular device, comprising: (a) a curved body 200, where the curved body comprises: (i) a first curved body 202 coupled to a second curved body 204; (ii) a magnet 206 disposed within the first curved body 202, the second curved body 204, or a combination of one or more thereof; and (iii) a third body 208 coupled to the first curved body 202, the second curved body 204, or a combination thereof, where the third body 208 extends orthogonally away from a surface of the first curved body 202, a surface of the second curved body 204, or a combination of one or more thereof, and where the third body 208 comprises a length of up to about a diameter of the first curved body 202 coupled to the second curved body 204; and (b) a magnetic field generator 116 providing an alternating magnetic field 117 to the magnet 206 disposed within the first curved body 202, the second curved body 204, or a combination of one or more thereof, thereby vibrating the first curved body 202, the second curved body 204, or a combination of one or more thereof, where the curved body 200 is provided to a blood vessel 118 of a subject, and where the magnetic field generator 116 is external to the blood vessel of the subject. In some cases, the first curved body 202, the second curved body 204, or a combination of one or more thereof, may comprise a hemisphere curved body. In some cases, the first curved body 202, the second curved body 204, the third body 208, or a combination of one or more thereof, may not be coupled to a guidewire. In some cases, the magnet 206 may be fixed within the first curved body 202, the second curved body 204, or a combination of one or more thereof.

In some instances, the third body 208 may comprise a tail of the first curved body 202, the second curved body 204, or a combination of one or more thereof. In some cases, the third body 208 may be coupled to the first curved body 202, the second curved body 204, or a combination of one or more thereof, at an apex of a curved surface of the first curved body 202, the second curved body 204, or a combination of one or more thereof. In some cases, the alternating magnetic field may comprise a rotating and/or oscillating magnetic field, as shown in FIGS. 12A and 12B, and where when the rotating and/or oscillating magnetic field is applied to the magnet 206, the third body 208 may propel and/or translate the first curved body 202, the second curved body 204, and the magnet 206 within the blood vessel of the subject.

In some cases, the first curved body 202, the second curved body 204, or a combination of one or more thereof, may be made of a biocompatible material. The biocompatible material may comprise a biocompatible plastic or silicon material.

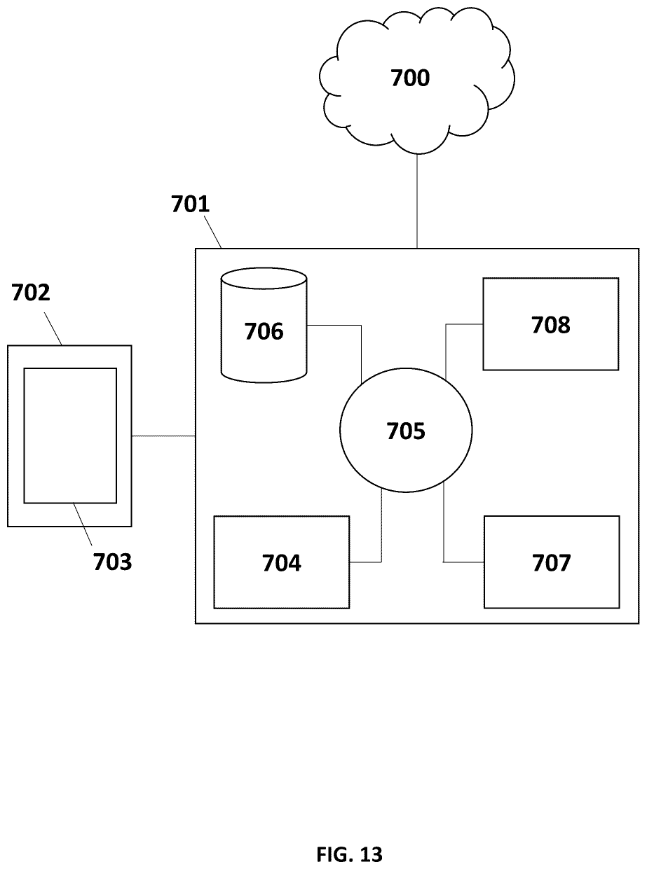

FIG. 13 shows a computer system 701 suitable for interfacing, controlling, communicating, and/or actuating, one or more intravascular devices and/or system sub-components (e.g., the magnetic field generator 116), described elsewhere herein. In some cases, the computer system 701 may control and/or actuate, a position, angle, and/or orientation of one or more mechanical arms coupled to one or more magnetic field generators 116, described elsewhere herein. In some cases, the computer system 701 may implement one or more methods (e.g., 800, 900, and/or 1000), described elsewhere herein. The computer system 701 may process various aspects of information and/or data generated by the systems and methods described elsewhere herein, such as, for example, a position of one or more intravascular devices (100, 200, 300, 400, 500, and/or 600) within a blood vessel 118 of a subject, with respect to a region and/or location of intravascular plaque 120.

In some cases, the computer system 701 may be electrically coupled and/or in electrical communication with an imaging system that visualizes the one or more intravascular devices (100, 200, 300, 400, 500, and/or 600) and/or a position of blood vessel plaque 120 within the blood vessel 118 of the subject. In some cases, the computer system 701 may be electrically coupled and/or in electrical communication with a magnetic field generator 116 to generate and direct one or more magnetic fields 117, described elsewhere herein (e.g., FIGS. 12A-12C) to one or more intravascular devices (100, 200, 300, 400, 500, and/or 600).

The computer system 701 may be an electronic device. In some cases, the computer system 701 may comprise a laptop computer, personal computer, integrated computing device and/or system, or any combination of one or more thereof. In some cases, the computer system 701 may comprise a computer system external to the systems, described elsewhere herein. In some cases, the computer system 701 may be a mobile electronic device in electrical communication with the system through a cable and/or over the internet and/or an intranet. The computer system 701 may comprise one or more central processing unit(s) (CPU, also “processor” and “computer processor” herein) 705, which may be a single core and/or a multi core processor, or a plurality of processor for parallel processing. In some cases, the processor may comprise a microcontroller. The computer system 701 may further comprise memory and/or memory locations 704 (e.g., random-access memory, read-only memory, and/or flash memory), electronic storage unit 706 (e.g., hard disk), communications interface 708 (e.g., network adapter) for communicating with one or more other devices, peripheral devices 707, such as cache, other memory, data storage and/or electronic display adapters, or any combination of one or more thereof. The memory 704, storage unit 706, interface 708, and peripheral devices 707 may be in communication with the CPU 705 through a communication bus (e.g., solid lines), such as a motherboard. The storage unit 706 may be a data storage unit (or a data repository) for storing data pertaining to known alternating magnetic field frequencies at the resonant frequency of the blood vessel plaque identified. The computer system 701 may be operatively coupled to a computer network (“network”) 700 with the aid of the communication interface 708. The network 700 may be the Internet, an internet and/or extranet, or an intranet and/or extranet that is in communication with the Internet. The network 700 may, in some case, be a telecommunication and/or data network. The network 700 may include one or more computer servers, which may enable distributed computing, such as cloud computing. The network 700, in some cases with the aid of the computer system 701, may implement a peer-to-peer network, which may enable devices coupled to the computer system 701 to behave as a client or a server.

The CPU 705 may execute a sequence of machine-readable instructions, which may be embodied in a program or software. The instructions may be directed to the CPU 705, which may subsequently program or otherwise configured the CPU 705 to implement methods of the present disclosure. Examples of operations performed by the CPU 705 may include fetch, decode, execute, and writeback.

The CPU 705 may be part of a circuit, such as an integrated circuit. One or more other components of the system 701 may be included in the circuit. In some cases, the circuit may be an application specific integrated circuit (ASIC).

The storage unit 706 may store files, such as drivers, libraries, and saved programs. The storage unit 706 may store imaging data of the one or more intravascular devices within the subject's blood vessel and/or imaging data of the blood vessel plaque. The computer system 701, in some cases may include one or more additional data storage units that are external to the computer system 701, such as located on a remote server that is in communication with the computer system 701 through an intranet or the internet 700.

Methods, as described herein, may be implemented by way of machine (e.g., computer processor) executable code stored on an electronic storage location of the computer device 701, such as, for example, on the memory 704 or electronic storage unit 706. The machine executable or machine-readable code may be provided in the form of software. During use, the code may be executed by the one or more processor(s) 705. In some instances, the code may be retrieved from the storage unit 706 and stored on the memory 704 for ready access by the one or more processor(s) 705. In some instances, the electronic storage unit 706 may be precluded, and machine-executable instructions are stored on memory 704.

The code may be pre-compiled and configured for use with a machine having a processor adapted to execute the code or may be compiled during runtime. The code may be supplied in a programming language that may be selected to enable the code to be executed in a pre-complied or as-compiled fashion.

Aspects of the systems and methods provided herein, such as the computer system 701, may be embodied in programming. Various aspects of the technology may be thought of a “product” or “articles of manufacture” typically in the form of a machine (or processor) executable code and/or associated data that is carried on or embodied in a type of machine readable medium. Machine-executable code may be stored on an electronic storage unit, such memory (e.g., read-only memory, random-access memory, flash memory) or a hard disk. “Storage” type media may include any or all of the tangible memory of a computer, processor the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage’ media, term such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media may include, for example, optical or magnetic disks, such as any of the storage devices in any computer(s) or the like, that may be used to implement the databases, etc. Volatile storage media may include dynamic memory, such as main memory of such a computer platform. Tangible transmission media may include coaxial cables, copper wire and fiber optics, including the wires that comprise a bus within a computer device. Carrier-wave transmission media may take the form of electric or electromagnetic signals, or acoustic or light waves such as those generated during radio frequency (RF) and infrared (IR) data communications. Common forms of computer-readable media may therefor include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one more instruction to a processor for execution.

The computer system may include or be in communication with an electronic display 702 that comprises a user interface (UI) 703 for setting and/or adjusting system parameters and/or settings e.g., the type of intravascular plaque and/or the intravascular procedure conducted, the type of intravascular plaque treated, the frequency of the alternating and/or rotating magnetic field, or any combination of one or more thereof settings. In some cases, the UI 703 may also comprise an interface where a medical professional, e.g., an interventional surgeon, operating room technician, nurse, etc., may operate the system and/or devices, described elsewhere herein. In some cases, a user of the system and/or the one or more intravascular devices may control providing and/or injecting the one or more intravascular devices to the subject, movement, and/or translation of the one or more intravascular devices to a target region and/or location within the subject's blood vessel to treat blood vessel plaque. Examples of UI's include, without limitation, a graphical user interface (GUI) and web-based user interface.

Methods and systems of the present disclosure, described elsewhere herein, can be implemented by way of one or more algorithms and with instructions provided with one or more processors as disclosed herein. An algorithm can be implemented by way of software upon execution by the central processing unit 705.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Although the above steps show each of the methods or sets of operations in accordance with embodiments described elsewhere herein, a person of ordinary skill in the art will recognize many variations based on the teaching described herein. The steps may be completed in a different order. Steps may be added or omitted. Some of the steps may comprise sub-steps. Many of the steps may be repeated as often as beneficial. One or more of the steps of each of the methods or sets of operations may be performed with circuitry as described herein, for example, one or more of the processor or logic circuitry such as programmable array logic for a field programmable gate array. The circuitry may be programmed to provide one or more of the steps of each of the methods or sets of operations, and the program may comprise program instructions stored on a computer readable memory or programmed steps of the logic circuitry such as the programmable array logic or the field programmable gate array, for example.

What is claimed is:

1. An intravascular device, comprising:
(a) a first curved body coupled to a rod at a first end of the rod, wherein a magnet is disposed within the first curved body;
(b) a mesh structure concentric with the rod, wherein the mesh structure comprises an inner surface and an outer surface;
(c) a deformable body adjacent to the inner surface of the mesh structure; and
(d) a second curved body coupled to a second end of the rod, wherein the second curved body is concentric with the rod, and wherein the second curved body applies a force to the deformable body thereby expanding the mesh structure when the second curved body is translated toward the first curved body at the first end of the rod.

2. The intravascular device of claim 1, wherein the first curved body, the second curved body, or a combination thereof, comprise a hemisphere curved body.

3. The intravascular device of claim 1, wherein the deformable body encapsulates a fluid, wherein the fluid comprises an osmolarity of about an osmolarity of mammalian blood or a density of about a density of mammalian blood.

4. The intravascular device of claim 1, wherein the mesh structure comprises a stent.

5. The intravascular device of claim 1, wherein the first curved body, the second curved body, or a combination thereof, comprises a flat surface configured to contact an external surface of the deformable body.

6. The intravascular device of claim 1, wherein a third body is coupled to the first curved body, the second curved body, or a combination thereof, at an apex of a curved surface of the first curved body, the second curved body, or a combination thereof, and wherein the third body extends orthogonally away from a surface of the first curved body, the second curved body, or a combination thereof.

7. The intravascular device of claim 1, wherein the second curved body comprises (i) an opening concentric with the rod, and (ii) a mounting interface disposed within the second curved body, wherein the mounting interface comprises an opening concentric or cordial with the opening of the second curved body, the rod, or a combination thereof.

8. The intravascular device of claim 1, wherein the deformable body comprises a material that changes from a first diameter to a second diameter when the second curved body applies the force to the deformable body, and wherein the first diameter is less than the second diameter.

9. The intravascular device of claim 1, wherein the first curved body, the second curved body, the rod, the mesh structure, the deformable body, or any combination thereof, are not coupled to a guide wire.

10. The intravascular device of claim 1, wherein a first pole and a second pole of the magnet are orthogonal to a central axis of the rod, and wherein the magnet is coupled to the rod such that it is concentric with a washer disposed within the first curved body.

11. The intravascular device of claim 1, wherein the third body comprises a helical body, a rigid body, a coil, a tail of the first curved body, a tail of the second curved body, or any combination thereof.

12. A method of fracturing intravascular plaque, comprising:
(a) vibrating a curved body in an inner lumen of a blood vessel of a subject with an alternating magnetic field provided external to the blood vessel, wherein the curved body comprises a magnet disposed within the curved body, wherein the curved body is not coupled to a guidewire, wherein the curved body contacts a surface of the inner lumen of the blood vessel adjacent to a blood vessel plaque, and wherein the curved body is vibrated at one or more frequencies in the blood vessel, wherein the one or more frequencies comprise one or more resonant frequencies of the blood vessel plaque; and
(b) fracturing the blood vessel plaque when the curved body contacts the surface of the inner lumen of the blood vessel adjacent to the blood vessel plaque,
wherein the fracturing increases compliance of the blood vessel.

13. The method of claim 12, wherein the curved body comprises a first curved body coupled to a second curved body, and wherein the first curved body and the second curved body are coupled to a rod such that the first curved body is disposed at a first end of the rod and the second curved body is disposed at a second end of the rod.

14. The method of claim 12, wherein the curved body comprises a first curved body coupled to a second curved body, wherein the first curved body, the second curved body, or a combination thereof, comprise a curved opening concentric with the rod comprising a region reversibly mated to a region of the surface of the rod.

15. The method of claim 13, wherein:
(i) the first curved body, the second curved body, or a combination thereof comprises a flat surface configured to contact an outer surface of the deformable body; and
(ii) an outer surface of the deformable body is in contact with a mesh structure concentric with the rod.

16. The method of claim 12, comprising applying a rotating magnetic field along an axis concentric with the rod thereby translating the second curved body toward the first curved body.

17. The method of claim 12, wherein the alternating magnetic field comprises an alternating magnetic field in a first dimension, a second dimension, a third dimension, or any combination thereof, wherein the first dimension, the second dimension, the third dimension, or any combination thereof, are orthogonal dimensions to each other.

18. The method of claim 13, further comprising a third body coupled to the curved body, the first curved body, the second curved body, or a combination thereof, wherein the third body is configured to:
(i) extend orthogonally away from a surface of the curved body, the first curved body, the second curved body, or a combination thereof;
(ii) couple to the curved body, the first curved body, the second curved body, or a combination thereof, at an apex of a curved cross-section of the curved body, a curved cross-section of the first curved body, a curved cross-section of the second curved body, or a combination thereof; and/or
(iii) maintain an orientation of the curved body, the first curved body, the second curved body, or a combination thereof, in the blood vessel of the subject with respect to the alternating magnetic field.

19. The method of claim 12, further comprising obtaining or receiving data of the blood vessel plaque volume, morphology, location, or any combination thereof, prior to or after fracturing the blood vessel plaque.

20. The method of claim 12, further comprising providing a magnetic field to translate or transport the curved body from a first location in the blood vessel of the subject to a second location in the blood vessel of the subject, wherein the first location and the second location do not spatially overlap.
